(19) United States
(12) Patent Application Publication
(10) Pub. No.: US 2023/0233389 A1
(43) Pub. Date: Jul. 27, 2023

(54) PATIENT SUPPORT APPARATUS SYSTEMS WITH DYNAMIC CONTROL ALGORITHMS

(71) Applicant: Stryker Corporation, Kalamazoo, MI (US)

(72) Inventors: Krishna Sandeep Bhimavarapu, Kalamazoo, MI (US); Anish Paul, Portage, MI (US); Marco N. Kostic, Oshawa, Ontario (CA); Sujay Sukumaran, Portage, MI (US); Marco Constant, Portage, MI (US); Alexey Titov, Redmond, WA (US)

(21) Appl. No.: 18/008,866

(22) PCT Filed: Sep. 13, 2021
(86) PCT No.: PCT/US21/49987

§ 371 Date: Dec. 7, 2022

Related U.S. Application Data
(60) Provisional application No. 63/077,864, filed on Sep. 14, 2020.

(51) Int. Cl. A61G 7/018; A61G 7/07; A61G 7/057; A61G 7/05; A61G 1/04; G16H 40/67

(52) U.S. Cl. CPC A61G 7/018; A61G 7/07; A61G 7/05769; A61G 7/0524; A61G 7/0527; A61G 1/04; G16H 40/67; A61G 2203/10; A61G 2203/32; A61G 2203/42; A61G 2203/30

(57) ABSTRACT

A patient support apparatus, such as a bed, stretcher, cot, or the like, includes a frame, a support surface, a control, a controller, and a transceiver. The patient support apparatus employs one or more machine learning techniques to perform one or more of the following: automatically implement one or more user-preferred settings, automatically predict the occurrence of one or more events based on analyses of prior events, and/or automatically improve one or more algorithms based on analyses of additional sensor data. The machine learning techniques may be implemented onboard the patient support apparatus and/or may be implemented at a remote computer device (e.g. a server) that collates and analyzes data from multiple patient support apparatuses, and then sends the results of the analyses back to the patient support apparatuses.

### CROSS-REFERENCE TO RELATED APPLICATIONS

This application claims priority to U.S. provisional patent application Ser. No. 63/077,864 filed Sep. 14, 2020, by inventors Krishna S. Bhimavarapu et al. and entitled PATIENT SUPPORT APPARATUS SYSTEMS WITH DYNAMIC CONTROL ALGORITHMS, the complete disclosure of which is incorporated herein by reference.

### BACKGROUND

The present disclosure relates to patient support apparatuses, such as beds, stretchers, cots, recliners, and the like, and more particularly to improved control systems for such patient support apparatuses.

Medical facilities, such as hospitals, typically include a plurality of patient support apparatuses that allow patients to rest thereon. Such patient support apparatuses typically include one or more control panels that enable the user to execute one or more functions of the patient support apparatus, such as, but not limited to, moving components of the patient support apparatus, taking weight readings, arming/disarming an exit detection system, arming/disarming a monitoring system, locking out one or more controls, communicating with offboard devices (e.g. a LAN), turning on/off one or more lights, configuring one or more aspects of the patient support apparatus, etc. Some of these functions are carried out in different manners, depending upon one or more settings that that can be selected by the user. Some of these functions may also be carried out by one or more algorithms that rely on data from one or more sensors, and the collection of sensor(s) used by the algorithm does not change over time.

### SUMMARY

A patient support apparatus system according to one or more embodiments of the present invention provides patient support apparatuses that are adapted to use machine learning to improve their operation over time. Such improvements may relate to automatically selecting one or more user-preferred settings after gathering data from previous selections by users. Additionally or alternatively, such improvements may relate to improving the prediction of one or more events and/or improving one or more algorithms by using additional sensor data that is determined to have a predictable influence on the function(s) carried out by the algorithm. In some embodiments, the predicted event and/or improved algorithm relate to any one or more of the following: a patient developing bed sores, a patient exiting the support surface, a patient developing ventilator associated pneumonia, and/or a user making a selection of one or more user-preferred settings associated with a function of the patient support apparatus.

A patient support apparatus according to one embodiment of the present disclosure includes a frame, a support surface, a control, a controller, and a transceiver. The support surface is adapted to support a patient thereon and is supported on the frame. The control is adapted to be activated by a user of the patient support apparatus. The controller communicates with the control and is adapted to carry out a function of the patient support apparatus in response to the control being activated by the user. The function is a type of function that is capable of being performed in a plurality of different manners based upon a setting selectable by the user. The controller records over time setting data indicating the particular setting selected by the user when the function is carried out. The transceiver is adapted to transmit the setting data to a computing device located off-board the patient support apparatus, which in turn is adapted to analyze the setting data and to determine a user-preferred setting when the function is carried out. The controller is further adapted to receive a message back from the computing device indicating the user-preferred setting and to automatically select the user-preferred setting in response to future activations of the control by the user.

According to other aspects of the present disclosure, the function carried out by the patient support apparatus is an exit alert issued when the patient moves toward exiting from the support surface, and the setting is a sensitivity level for the exit alert.

In some embodiments, the support surface includes a backrest section adapted to pivot over a range of orientations, and the function is a lockout function adapted to prevent pivoting of the backrest section below a specific angle. In such embodiments, the setting is the specific angle.

In some embodiments, the patient support apparatus further includes an inflatable bladder in communication with the controller. In such embodiments, the function may be a therapy function administered by the inflatable bladder and the setting may be at least one of a duration, a frequency, or a rotational angle related to the therapy function.

In some embodiments, the patient support apparatus further comprises at least one sensor and the controller is further adapted to take multiple sets of readings from the sensor and record the sets of readings. Each set of the multiple sets of readings including readings taken both before and after an occurrence of an event associated with the patient support apparatus. The controller is further adapted to transmit the sets of readings to the computing device. In some embodiments, the controller is further adapted to receive an algorithm back from the computing device for predicting a future occurrence of the event.

In some embodiments, the patient support apparatus further comprises a plurality of sensors and the controller is further adapted to take a set of readings from the plurality of sensors, record the set of readings, and use a first subset of the set of readings in an algorithm for carrying out the function of the patient support apparatus. In such embodiments, the first subset excludes readings from at least one sensor in the plurality of sensors and the controller is further adapted to transmit the set of readings to the computing device.

The controller may further be adapted to receive an improved algorithm back from the computing device and to use the improved algorithm when carrying out the function. The improved algorithm may use a second subset of the readings from the plurality of sensors, wherein the second subset is different from the first subset.

In some embodiments, the second subset of the readings does not exclude readings from the at least one sensor in the plurality of sensors.

In some embodiments, the first subset includes at least one reading from a sensor not included in the second subset.

A patient support apparatus system according to another embodiment of the present disclosure includes a patient support apparatus and a remote computing device. The patient support apparatus includes a frame, a support surface adapted to support a patient thereon, a sensor, a controller, and a transceiver. The controller is adapted to take multiple sets of readings from the sensor and record the sets of readings, wherein each set of the multiple sets of readings including readings taken both before and after an occurrence of an event associated with the patient support apparatus. The computing device is in communication with the transceiver and adapted to receive the sets of readings from the patient support apparatus and to analyze the sets of readings to determine an algorithm for predicting a future occurrence of the event using future readings from the sensor.

According to other aspects of the present disclosure, the event is the patient exiting from the support surface and the sensor comprises a plurality of force sensors adapted to detect downward forces exerted on the support surface.

In some embodiments, the event is the patient contracting ventilator associated pneumonia, the support surface includes a backrest section adapted to pivot over a range of orientations, and the sensor is adapted to measure an angular orientation of the backrest section.

In some embodiments, the event is the patient contracting a bed sore, the patient support apparatus includes an inflatable bladder in communication with the controller and adapted to administer a therapy function to the patient, and the sensor is adapted to measure at least one of a duration, a frequency, or a rotational angle related to the therapy function.

In some embodiments, the controller is further adapted to execute a function using the sensor and a second algorithm.

In some embodiments, the patient support apparatus further comprises a plurality of additional sensors, and the controller is adapted to receive an additional set of readings from the additional sensors, to record the additional set of readings, and to transmit the additional set of readings to the computing device. In some embodiments, the controller is still further adapted to receive an improved second algorithm back from the computing device and to use the improved second algorithm when carrying out the function. The improved second algorithm uses readings from at least one of the plurality of additional sensors.

In some embodiments, the plurality of additional sensors includes at least two of the following: a siderail sensor adapted to detect a position of a siderail of the patient support apparatus, an angle sensor adapted to detect an angle of a pivotable backrest section of the patient support apparatus, an angle sensor adapted to detect an angle of a litter frame on which the support surface is supported, a light sensor adapted to detect an amount of ambient light in a room in which the patient support apparatus is positioned, a sound sensor adapted to detect an amount of sound in the room in which the patient support apparatus is positioned, or a clock adapted to detect a current time.

In some embodiments, the computing device is adapted to use a neural network to generate the improved second algorithm. The computing device may use at least two of the following as inputs into the neural network: a patient fall history, a patient weight, a patient age, a patient fall risk assessment, a time of day, an amount of time since the patient last exited from the patient support apparatus, and a calendar date.

In some embodiments, the controller is further adapted to perform a function of the patient support apparatus when a control is activated by a user, and the function is performable in a plurality of different manners based upon a setting selectable by the user. In such embodiments, the event may be the selection of the setting by the user, and that the algorithm may be adapted to automatically predict a future setting in response to the user activating the control, thereby relieving the user of the task of having to manually select the setting.

A patient support apparatus system according to another embodiment of the present disclosure includes a plurality of patient support apparatuses and a remote computing device. Each of the patient support apparatuses includes a frame, a support surface adapted to support a patient thereon, a plurality of sensors, a controller, and a transceiver. The controller is adapted to take a set of readings from the plurality of sensors, record the set of readings, and use a first subset of the set of readings in an algorithm for carrying out a function of the patient support apparatus. The first subset excludes readings from at least one sensor in the plurality of sensors. The computing device is adapted to receive the set of readings from the plurality of patient support apparatuses and to analyze the set of readings to determine an improved algorithm for use by each of the plurality of patient support apparatuses when carrying out the function.

According to other aspects of the present disclosure, the controller may further be adapted to receive the improved algorithm back from the computing device and to use the improved algorithm when carrying out the function. The improved algorithm uses a second subset of the readings from the plurality of sensors and the second subset is different from the first subset. In some embodiments, the second subset of the readings does not exclude readings from the at least one sensor in the plurality of sensors. Alternatively or additionally, the first subset may include at least one reading from a sensor not included in the second subset.

In some embodiments, the support surface includes a backrest section adapted to pivot over a range of orientations, the function is a lockout function adapted to prevent pivoting of the backrest section below a specific angle, and the first subset includes readings from an angle sensor adapted to detect an angular orientation of the backrest section.

In some embodiments, each of the plurality of patient support apparatuses further comprises an inflatable bladder in communication with the controller. In such embodiments, the function may be a therapy function administered by the inflatable bladder and the first subset may include readings from an angle sensor adapted to detect a rotational angle related to the therapy function. The set of readings may include readings taken from at least one of a scale system adapted to detect a weight of the patient or a timer adapted to measure an amount of time since the therapy function was previously performed.

In some embodiments, the function is an exit alert issued when the patient moves toward exiting from the support surface, and the first subset includes reading from a plurality of force sensors adapted to detect downward forces exerted on the support surface. In such embodiments, the plurality of sensors may include at least two of the following: a siderail sensor adapted to detect a position of a siderail of a respective patient support apparatus, an angle sensor adapted to detect an angle of a pivotable backrest section of the respective patient support apparatus, an angle sensor adapted to detect an angle of a litter frame on which the support surface is supported, a light sensor adapted to detect an amount of ambient light in a room in which the respective patient support apparatus is positioned, a sound sensor adapted to detect an amount of sound in the room in which the respective patient support apparatus is positioned, or a clock adapted to detect a current time.

In some embodiments, the computing device is adapted to use a neural network to analyze the set of readings and determine the improved algorithm. The computing device may further be adapted to use at least two of the following as inputs into the neural network: a patient fall history, a patient weight, a patient age, a patient fall risk assessment, a time of day, an amount of time since the patient last exited from the support surface, and a calendar date. The computing device may still further be adapted to use two of the following as additional inputs the neural network: a position of a siderail of a respective patient support apparatus, an angle of a pivotable backrest section of the respective patient support apparatus, an angle of a litter frame on which the support surface is supported, an amount of ambient light in a room in which the respective patient support apparatus is positioned, or an amount of sound in the room in which the respective patient support apparatus is positioned.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration is used in the description herein of various embodiments (e.g. first, second, third, etc.). Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first embodiment of the disclosure;

FIG. 2 is a plan view of one of the control panels of the patient support apparatus of FIG. 1;

FIG. 3 is a block diagram of a control system of the patient support apparatus;

FIG. 4 is a flow diagram of an automatic setting selection algorithm that may be followed by the patient support apparatus;

FIG. 5 is an example of an exit detection control screen for an exit detection system of the patient support apparatus that is displayable on a display of the patient support apparatus;

FIG. 6 is an example of a motion lockout control screen that is displayable on a display of the patient support apparatus;

FIG. 7 is an example of a monitoring control screen for a monitoring system of the patient support apparatus that is displayable on a display of the patient support apparatus;

FIG. 8 is an example of a mattress therapy control screen that is displayable on a display of the patient support apparatus; and

FIG. 9 is an example of a new patient protocol screen that is displayable on a display of the patient support apparatus;

FIG. 10 is an example of a low height selection screen that is displayable on a display of the patient support apparatus;

FIG. 11 is a flow diagram of a future event prediction algorithm that may be followed by the patient support apparatus; and

FIG. 12 is a diagram of a neural network that may be used by the patient support apparatus and/or a computer device located off-board the patient support apparatus in order to improve an exit detection algorithm associated with the patient support apparatus.

### DETAILED DESCRIPTION OF THE EMBODIMENTS

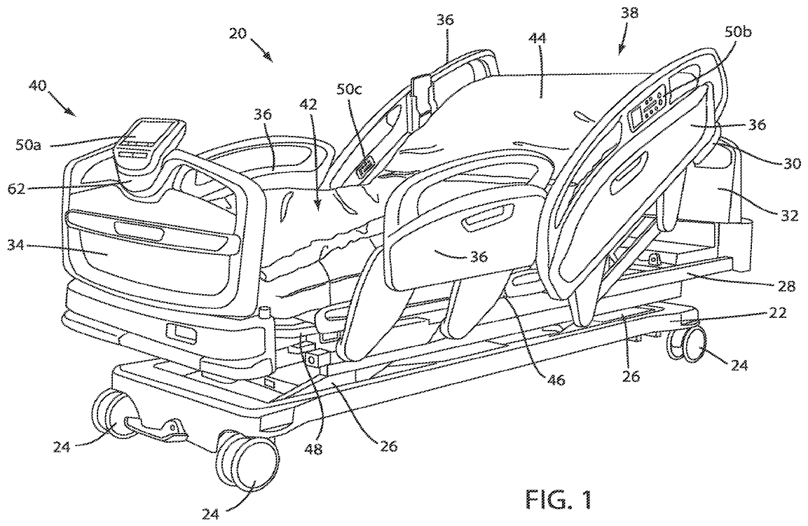

An illustrative patient support apparatus 20 according to a first embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, an operating table, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40. The lifts 26 may be constructed and/or operated in any of the manners disclosed in commonly assigned U.S. patent publication 2017/0246065, filed on Feb. 22, 2017, entitled LIFT ASSEMBLY FOR PATIENT SUPPORT APPARATUS, the complete disclosure of which is hereby incorporated herein by reference. Other manners for constructing and/or operating lifts 26 may, of course, be used.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. The top surface of the mattress 42 or other cushion forms a support surface for the occupant.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the caregiver, or other user of patient support apparatus 20, to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width. Manners in which deck 30 may be constructed in order to allow itself to be adjusted to different widths are disclosed in more detail in commonly assigned U.S. patent application Ser. No. 62/994,540 filed Mar. 25, 2020, by inventors Jason Connell et al. and entitled PATIENT SUPPORT WITH DECK WIDTH MONITORING AND CONTROL, the complete disclosure of which is incorporated herein by reference. Still other types of expandable width decks may be used.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions besides what is shown in FIG. 1, such as, but not limited to, those described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may be the same as, or nearly the same as, the mechanical construction of the Model 3002 S3 bed manufactured and sold by Stryker Corporation of Kalamazoo, Mich. This mechanical construction is described in greater detail in the Stryker Maintenance Manual for the MedSurg Bed, Model 3002 S3, published in 2010 by Stryker Corporation of Kalamazoo, Mich., the complete disclosure of which is incorporated herein by reference. It will be understood by those skilled in the art that patient support apparatus 20 can be designed with still other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of patient support apparatus 20 may also take on still other forms different from what is disclosed in the aforementioned references.

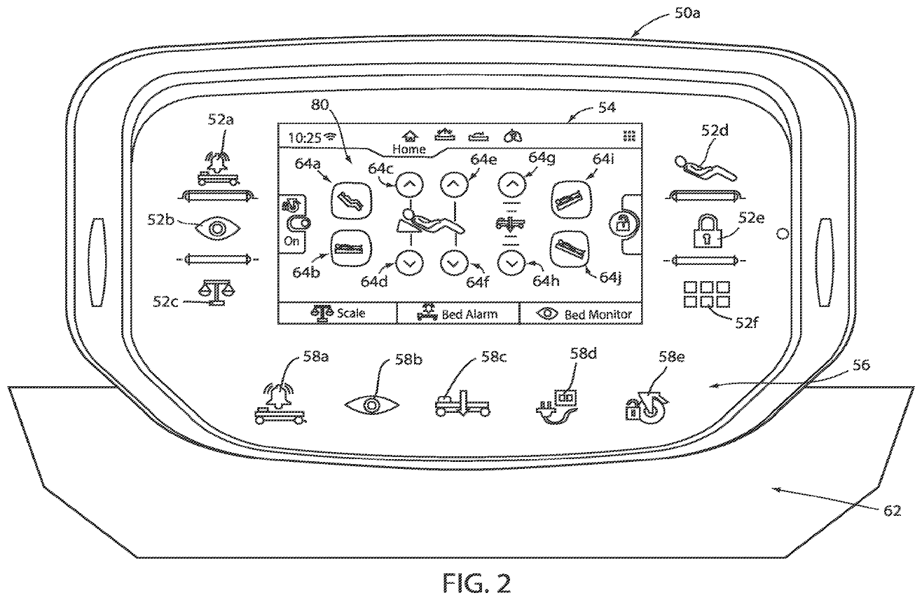

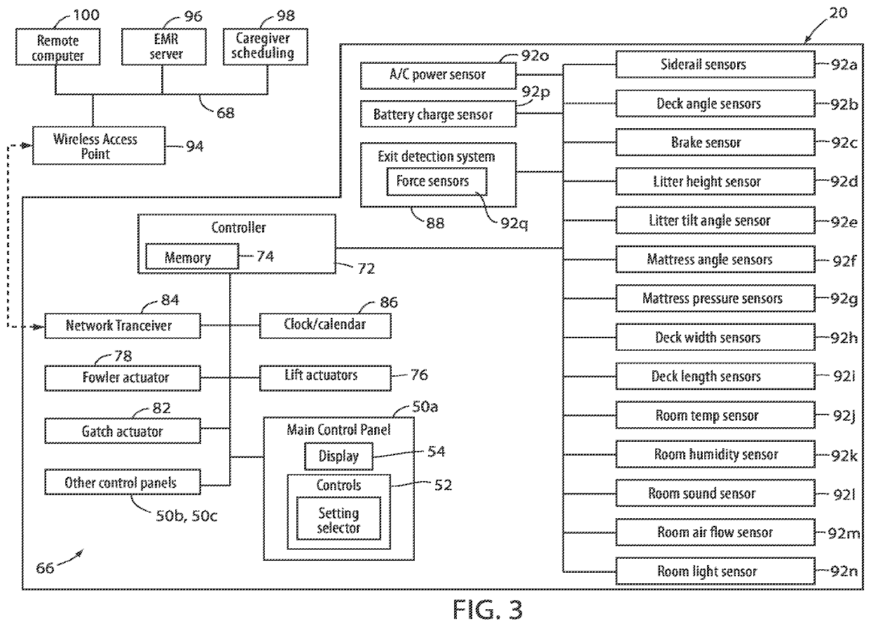

Patient support apparatus 20 further includes a plurality of control panels 50 that enable a user of patient support apparatus 20, such as a patient and/or an associated caregiver, to control one or more aspects of patient support apparatus 20. In the embodiment shown in FIG. 1, patient support apparatus 20 includes a footboard control panel 50a, a pair of outer siderail control panels 50b (only one of which is visible), and a pair of inner siderail control panels 50c (only one of which is visible). Footboard control panel 50a and outer siderail control panels 50b are intended to be used by caregivers, or other authorized personnel, while inner siderail control panels 50c are intended to be used by the patient associated with patient support apparatus 20. Each of the control panels 50 includes a plurality of controls 52 (see, e.g. FIGS. 2-3), although each control panel 50 does not necessarily include the same controls and/or functionality.

Among other functions, controls 52 of control panel 50a allow a user to control one or more of the following: change a height of support deck 30, raise or lower head section 44, activate and deactivate a brake for wheels 24, arm and disarm an exit detection system, and communicate with the particular IT infrastructure installed in the healthcare facility in which patient support apparatus 20 is positioned. One or both of the inner siderail control panels 50c also include at least one control that enables a patient to call a remotely located nurse (or other caregiver). In addition to the nurse call control, one or both of the inner siderail control panels 50c may also include one or more controls for controlling one or more features of a television, room light, and/or reading light positioned within the same room as the patient support apparatus 20. With respect to the television, the features that may be controllable by one or more controls 52 on control panel 50c include, but are not limited to, the volume, the channel, the closed-captioning, and/or the power state of the television. With respect to the room and/or night lights, the features that may be controlled by one or more controls 52 on control panel 50c include the on/off state of these lights.

Control panel 50a includes a display 54 (FIG. 2) configured to display a plurality of different screens thereon. Display 54 may be a touchscreen-type display, although it will be understood that a non-touchscreen display may alternatively be used. Display 54 displays one or more visual indicators, one or more controls, and/or one or more control screens, and/or other types of information, as will be discussed more below. Display 54 may comprise an LED display, an OLED display, or another type of display. Display 54 may be configured to have its brightness level adjusted. That is, the amount of light emitted from display 54 can be varied by a controller included within patient support apparatus 20. In some embodiments, the brightness is adjusted based on one or more ambient light sensors, such as is disclosed in commonly assigned U.S. patent application Ser. No. 63/031,973 filed May 29, 2020, by inventors Frank Lee et al. and entitled PATIENT SUPPORT APPARATUS WITH AUTOMATIC DISPLAY CONTROL, the complete disclosure of which is incorporated herein by reference. Still other types of brightness control, sensors, and/or sensing systems may be used.

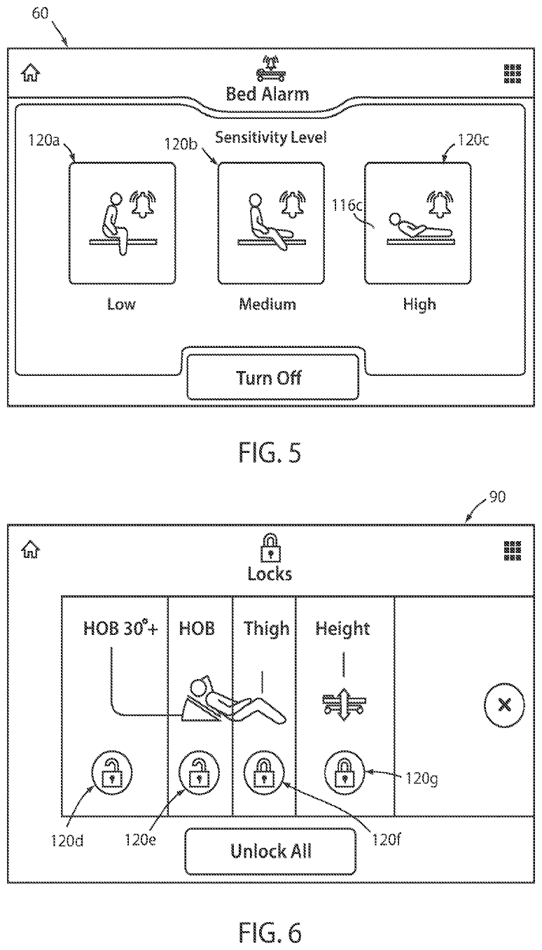

Surrounding display 54 are a plurality of navigation controls 52a-f that, when activated, cause the display 54 to display different screens on display 54. More specifically, when a user presses navigation control 52a, control panel 50a displays an exit detection control screen on display 54 that includes one or more icons that, when touched, control an onboard exit detection system. The exit detection system is as adapted to issue an alert when a patient exit from patient support apparatus 20. Such an exit detection system may include any of the features and functions as, and/or may be constructed in any of the same manners as, the exit detection system disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. Other types of exit detection systems can also or alternatively be used. As will be discussed in greater detail below, one example of an exit detection control screen 60 that may be displayed on display 54 in response to a user pressing on navigation control 52a is shown in FIG. 5.

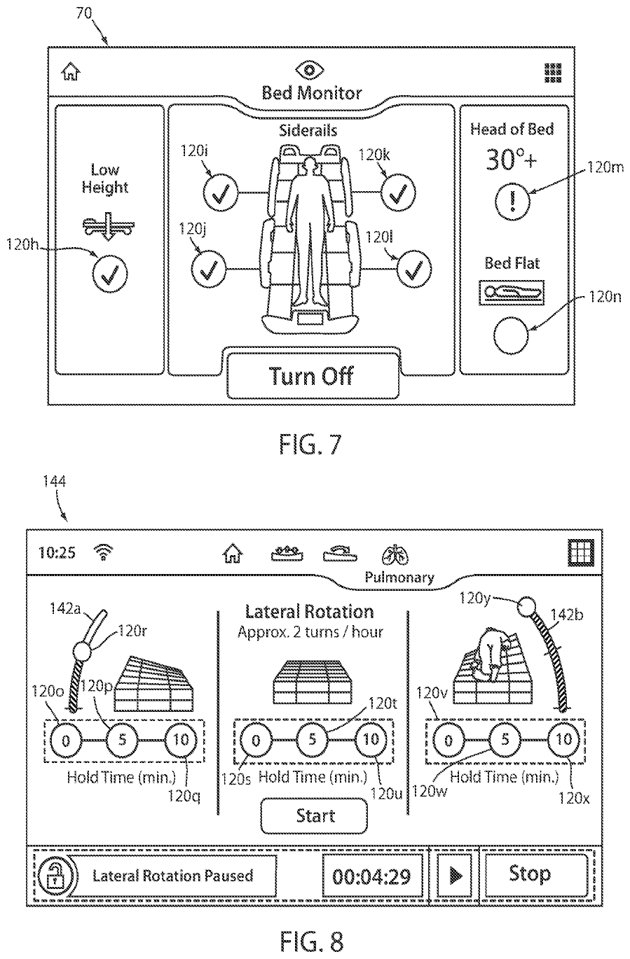

When a user presses navigation control 52b (FIG. 2), control panel 50 displays a monitoring control screen that includes a plurality of control icons that, when touched, control an onboard monitoring system built into patient support apparatus 20. One example of such a monitoring control screen 70 is shown in FIG. 7. Further details of monitoring screen 70 and the onboard monitoring system are provided below. Additional details may also be found in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems can also or alternatively be included with patient support apparatus 20.

When a user presses navigation control 52c, control panel 50a displays a scale control screen (not shown) that includes a plurality of control icons that, when touched, control the scale system of patient support apparatus 20. Such a scale system may include any of the features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. Other types of scale systems can also or alternatively be included with patient support apparatus 20.

When a user presses navigation control 52d, control panel 50 displays a motion control screen that includes a plurality of control icons that, when touched, control the movement of various components of patient support apparatus 20, such as, but not limited to, the height of litter frame 28 and the pivoting of head section 44. One example of such a motion control screen 80 is shown in FIG. 2. In some embodiments, the motion control screen displayed on display 54 in response to pressing control 52d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens can also or alternatively be included with patient support apparatus 20.

When a user presses navigation control 52e, control panel 50a displays a motion lockout control screen that includes a plurality of control icons that, when touched, control one or more motion lockout functions of patient support apparatus 20. One example of such a motion lockout control screen 90 is shown in FIG. 6. The motion lockout control screen 90 may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of motion lockout control screens can also or alternatively be included with patient support apparatus 20.

When a user presses on navigation control 52f, control panel 50a displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menu screens can also or alternatively be included with patient support apparatus 20.

For all of the navigation controls 52a-f (FIG. 2), screens other than the ones specifically mentioned above may be displayed on display 54 in other embodiments of patient support apparatus 20 in response to a user pressing these controls. Thus, it will be understood that the specific screens mentioned above are merely representative of the types of screens that are displayable on display 54 in response to a user pressing on one or more of navigation controls 52a-f. It will also be understood that, although navigation controls 52a-f have all been illustrated in the accompanying drawings as dedicated controls that are positioned adjacent display 54, any one or more of these controls 52a-f could alternatively be touchscreen controls that are displayed at one or more locations on display 54. Still further, although controls 52a-f have been shown herein as buttons, it will be understood that any of controls 52a-f could also, or alternatively, be switches, dials, or other types of non-button controls.

Control panel 50a, in some embodiments, also includes a dashboard 56 (FIG. 2) that communicates the current states of various conditions of patient support apparatus 20 to a caregiver. Dashboard 56 comprises a plurality of icons 58 that are individually illuminated via icon lights to thereby act as visual indicators for indicating the current state of different conditions of patient support apparatus 20. For example, as shown more clearly with respect to FIG. 2, a first icon 58a (e.g., a graphical symbol of an alert over a bed) is backlit (or backlit in a first color—e.g. green) by a backlight when an exit detection system is armed; a second icon 58b (e.g., a graphical symbol of an eye) is backlit (or backlit in a first color—e.g. green) when a monitoring system is armed; a third icon 58c (e.g., a graphical symbol of an arrow and bed) is backlit (or backlit in a first color—e.g. green) when litter frame 28 is at its lowest height (or below a threshold height); a fourth icon 58d (e.g., a graphical symbol of an unplugged AC power cord) is backlit (or backlit in a first color—e.g. green) when the patient support apparatus 20 is plugged into an electrical wall outlet; and a fifth icon 58e (e.g., a graphical symbol of a lock and wheel) is backlit (or backlit in a first color—e.g. green) when the brake is activated. When any of these conditions are not met (e.g. the exit detection system is unarmed, the monitoring system is unarmed or detecting an undesired condition, the litter frame is not at its lowest height, the AC power cord is not plugged in, or the brake is not activated, these icons 58a-e are backlit in a different color (e.g. amber), not backlit, or otherwise changed in appearance in order to convey these unmet conditions to the caregiver. Alternatively, any of icons 58a-e may be dead fronted (e.g. unlit and essentially not visible) when the corresponding condition is met and illuminated when the condition is not met. Additionally, fewer or additional icons 58 may be included as part of dashboard 56 (FIG. 2).

Still referring to FIG. 2, one or more reflective surfaces 62 may be located on the patient support apparatus 20 proximate control panel 50a. The reflective surfaces 62 may be disposed relative to control panel 50a such that one or more indirect lights (not shown) supported by the housing of control panel 50a project light away from control panel 50a toward the reflective surfaces 62 to be reflected off the reflective surfaces 62 and outward from the patient support apparatus 20 to act as another visual indicator of the current state of patient support apparatus 20. One example of such indirect lights and their operation are described in more detail in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, the complete disclosure of which is incorporated herein by reference. Other types of indirect lights, or no indirect lights, may also or alternatively be used with patient support apparatus 20.

FIG. 2 illustrates one example of a motion control screen 80 that is displayable on display 54 of patient support apparatus 20. Motion control screen 80 is displayed in response to a user navigating to it, such as by pressing on navigation control 52d. In some embodiments, motion control screen 80 may be the default screen which is initially displayed on display 54 and/or it may be the screen to which display 54 automatically returns after a predetermined time period of inactivity.

Motion control screen 80 includes a plurality of motion controls 64 for controlling the movement of patient support apparatus 20. Specifically, it includes a chair control 64a for moving patient support apparatus 20 to a chair configuration; a flat control 64b for moving patient support apparatus 20 to a flat orientation; a set of Fowler lift and lower controls 64c and 64d; a set of gatch lift and lower controls 64e and 64f; a litter frame lift control 64g; a litter frame lower control 64h; a Trendelenburg control 64i; and a reverse Trendelenburg control 64j. In some embodiments of patient support apparatus 20, motion control screen 80 are dedicated controls that are separate from display 54.

FIG. 3 illustrates in greater detail a control system 66 onboard patient support apparatus 20, as well as a local area network 68 that may be installed in a healthcare facility in which patient support apparatus 20 is located. Control system 66 of patient support apparatus 20 includes a controller 72, a memory 74, a plurality of lift actuators 76, a Fowler actuator 78, a gatch actuator 82, a network transceiver 84, a main control panel 50a, one or more additional control panels 50b, 50c, a clock/calendar 86, an exit detection system 88, and a plurality of sensors 92a-q. It will be understood by those skilled in the art that patient support apparatus 20 may be modified to include additional components not shown in FIG. 3, as well modified to include fewer components from what is shown in FIG. 3.

Controller 72 (FIG. 3) is constructed of any electrical component, or group of electrical components, that are capable of carrying out the functions described herein. In many embodiments, controller 72 is a conventional microcontroller, or group of conventional microcontrollers, although not all such embodiments need include a microcontroller. In general, controller 72 includes any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units as part of an embedded network. When implemented to include an embedded network, the embedded network may include multiple nodes that communicate using one or more of the following: a Controller Area Network (CAN); a Local Interconnect Network (LIN); an I-squared-C serial communications bus; a serial peripheral interface (SPI) communications bus; any of RS-232, RS-422, and/or RS-485 communication interfaces; a LonWorks network, and/or an Ethernet. The instructions followed by controller 72 in carrying out the functions described herein, as well as the data necessary for carrying out these functions, are stored in memory 74, and/or in one or more other memories accessible to the one or more microprocessors, microcontrollers, or other programmable components of controller 72. Memory 74 also includes a unique ID that uniquely identifies the particular patient support apparatus into which it is incorporated, such as, but not limited to, a serial number.

When controller 72 is implemented to communicate using an on-board Ethernet, the on-board Ethernet may be designed in accordance with any of the Ethernet-carrying patient support apparatuses disclosed in commonly assigned U.S. patent application Ser. No. 14/622,221 filed Feb. 13, 2015, by inventors Krishna Bhimavarapu et al. and entitled COMMUNICATION METHODS FOR PATIENT HANDLING DEVICES, the complete disclosure of which is incorporated herein by reference. In some embodiments, controller 72 may be implemented to include multiple nodes that communicate with each other utilizing different communication protocols. In such embodiments, controller 72 may be implemented in accordance with any of the embodiments disclosed in commonly assigned U.S. patent application Ser. No. 15/903,477 filed Feb. 23, 2018, by inventors Krishna Bhimavarapu et al. and entitled PATIENT CARE DEVICES WITH ON-BOARD NETWORK COMMUNICATION, the complete disclosure of which is incorporated herein by reference.

Lift actuators 76 (FIG. 3) are components of lifts 26 and are configured to raise and lower litter frame 28 with respect to base 22. A first one of lift actuators 76 powers a first one of the lifts 26 positioned adjacent head end 38 of patient support apparatus 20 and a second one of lift actuators 76 powers a second one of the lifts 26 positioned adjacent foot end 40 of patient support apparatus 20. Lift actuators 76 may be conventional linear actuators having electric motors therein that, when driven, expand or contract the length of the linear actuator, thereby moving the litter frame 28 upward or downward and changing its height relative to the floor.

Each lift actuator 76 includes a corresponding lift sensor that detects a position and/or angle of its associated actuator 76 and feeds the sensed position/angle to controller 72. Controller 72 uses the outputs from these sensors as inputs into a closed-loop feedback system for controlling the motion of the actuators 76 and the litter deck. Controller 72 also uses the outputs from these sensors as a litter height sensor 92d and a litter tilt angle sensor 92e. That is, controller 72 concludes that the litter angle is zero (horizontal or parallel to the floor) if the outputs from each of these sensors is the same, or nearly identical. If they are not the same or nearly identical, controller 72 is configured to calculate the litter angle based on the magnitude of the difference between these two sensors. Still further, controller 72 is configured to calculate the litter height from the outputs of these two sensors. In some embodiments, actuators 76 are constructed in any of the same manners as the actuators 34 disclosed in commonly assigned U.S. patent application Ser. No. 15/449,277 filed Mar. 3, 2017, by inventors Anish Paul et al. and entitled PATIENT SUPPORT APPARATUS WITH ACTUATOR FEEDBACK, the complete disclosure of which is incorporated herein by reference. In such embodiments, the internal sensors may be constructed to include any of the encoders and/or switch sensors disclosed in the aforementioned '277 application.

Fowler actuator 78 and gatch actuator 82 may be constructed in the same manner as lift actuators 76, or they may be constructed in different manners. Fowler actuator 78 is adapted to pivot Fowler section 44 upward or downward when it is driven. Gatch actuator 82 is adapted to pivot upward or downward the joint between thigh section 46 and a seat section portion of support deck 30 when it is driven such that the patient's knees are lifted or lowered.

Controller 72 communicates with network transceiver 84 (FIG. 3) which, in at least one embodiment, is a Wi-Fi radio communication module configured to wirelessly communicate with one or more wireless access points 94 of local area network 68. In such embodiments, network transceiver 84 may operate in accordance with any of the various IEEE 802.11 standards (e.g. 802.11b, 802.11n, 802.11g, 802.11ac, 802.11ah, etc.). In other embodiments, network transceiver 84 may include, either additionally or in lieu of the Wi-Fi radio and communication module, a wired port for connecting a network wire to patient support apparatus 20. In some such embodiments, the wired port accepts a category 5e cable (Cat-5e), a category 6 or 6a (Cat-6 or Cat-6a), a category 7 (Cat-7) cable, or some similar network cable, and transceiver 84 is an Ethernet transceiver. In still other embodiments, network transceiver 84 may be constructed to include the functionality of the communication modules 56 disclosed in commonly assigned U.S. patent application Ser. No. 15/831,466 filed Dec. 5, 2017, by inventor Michael Hayes et al. and entitled NETWORK COMMUNICATION FOR PATIENT SUPPORT APPARATUSES, the complete disclosure of which is incorporated herein by reference.

Regardless of the specific structure included with network transceiver 84, controller 72 is able to communicate with the local area network 68 (FIG. 3) of a healthcare facility in which the patient support apparatus 20 is positioned. When network transceiver 84 is a wireless transceiver, it communicates with local area network 68 via one or more wireless access points 94. When network transceiver 84 is a wired transceiver, it communicates directly via a cable coupled between patient support apparatus 20 and a network outlet positioned within the room of the healthcare facility in which patient support apparatus 20 is positioned.

Local area network 68 typically includes a plurality of servers, the contents of which will vary from healthcare facility to healthcare facility. In general, however, most healthcare facilities will include, at a minimum an electronic medical records (EMR) server 96 and a caregiver scheduling server 98. Such servers 96 and 98 may be conventional servers. In addition to these servers, local area network 68 includes a remote computer 100 or remote computer 100 (the two terms are used interchangeably herein) that is in communication with a plurality of patient support apparatuses 20 positioned within the healthcare facility. Remote computer 100 may also be communicatively coupled (via the Internet or other means) to one or more other servers that are positioned outside of the healthcare facility.

In addition to the aforementioned servers 96, 98, and 100, one or more additional servers may also be included, such as, but not limited to, an Internet server and/or an Internet gateway that couples network 68 to the Internet, thereby enabling remote computer 100, patient support apparatuses 20, and/or other applications on network 68 to communicate with computers outside of the healthcare facility, such as, but not limited to, a geographically remote server operated under the control of the manufacturer of patient support apparatuses 20. Another type of server that may be included with computer network 68 is a location server (not shown) that is adapted to monitor and record the current locations of patient support apparatuses 20, patients, and/or caregivers within the healthcare facility. Such a location server communicates with the patient support apparatuses 20 via access points 94 and network transceivers 84.

Network 68 may also include a conventional Admission, Discharge, and Tracking (ADT) server that allows patient support apparatuses 20 to retrieve information identifying the patient assigned to a particular patient support apparatus 20. Still further, healthcare network 68 may further include one or more conventional work flow servers and/or charting servers that assign, monitor, and/or schedule patient-related tasks to particular caregivers, and/or one or more conventional communication servers that forward communications to particular individuals within the healthcare facility, such as via one or more portable devices (smart phones, pagers, beepers, laptops, etc.). The forwarded communications may include data and/or alerts that originate from patient support apparatuses 20 and/or elsewhere.

In some embodiments, local area network 68 may include any and/or all of the servers described and disclosed in commonly assigned PCT patent application serial number PCT/US2020/039587 filed Jun. 25, 2020, by inventors Thomas Durlach et al. and entitled CAREGIVER ASSISTANCE SYSTEM, the complete disclosure of which is incorporated herein by reference. Further, in in such embodiments, patient support apparatus 20 may be configured to communicate with the servers on LAN 68 in any of the manners disclosed in the '587 PCT application, and/or to retrieve and/or share any of the information disclosed in the '587 PCT application.

Control system 66 includes a clock/calendar 86 (FIG. 3) that communicates with controller 72. Clock/calendar 86 not only measures the passage of time, but it also keeps track of the calendar day (and year). As will be discussed in greater detail below, controller 72 may use the outputs from clock/calendar day when it gathers data for improving one or more algorithms followed by controller 72, and/or when it automatically implements one or more user-preferred settings. Clock/calendar 86 may be any conventional timing device that is able to keep track of the passage of time, including the calendar day and year.

As was noted, controller 72 receives information from a plurality of sensors 92a-q positioned onboard patient support apparatus 20. Each of these sensors will now be described in greater detail.

Siderail sensors 92a (FIG. 3) are adapted to determine what position each siderail 36 currently is in (raised, lowered, or, in some embodiments, one or more intermediate positions between the raised and lowered positions).

Deck angle sensors 92b are adapted to determine the angular position of each section of deck 30 that is able to be pivoted. Deck angle sensors 92b therefore include a Fowler angle sensor that measure the current angular orientation of head section 44 (also known as a Head of Bed (HOB) angle) and a thigh and/or foot section sensor that measure the current angular orientation of thigh section 46 and/or foot section 48. In some embodiments of patient support apparatus 20 that include a pivotable seat section, a corresponding deck angle sensor 92b may be included that measures a current angular orientation of the seat section (with respect to a known reference, such as, but not limited to, for example, a horizontal or vertical reference).

Brake sensor 92c is adapted to determine whether a brake has been applied to one or more of the wheels 24 of patient support apparatus 20. In some embodiments, the brake is a mechanical brake that is movable between a braked position and an unbraked position, and brake sensor 82c is adapted to detect which position the brake currently is in. In other embodiments, the brake may be implemented in an electrical or other manner, and the brake sensors 92c may be implemented in one or more different manners.

Litter height sensor 92d and litter tilt angle sensor 92e were previously discussed and the former measures the height of litter frame 28 and the latter measures the tilt of litter frame 28 (e.g. with respect to horizontal or a component of patient support apparatus 20 that is typically horizontal). Sensors 92d and 92e may directly measure one or both of these quantities or they may be implemented, as discussed above, as sensors that detect the current position of lifts 26, thereby enabling controller 72 to determine the current height and tilt of litter frame 28. Still other manners of implementing these sensors 92d and/or 92e may be used.

Mattress angle sensors 92f are adapted to determine the angular orientation of one or more portions of an inflatable mattress 42 positioned on top of support deck 30. Mattress angle sensors 92f are typically incorporated into a mattress 42 and in communication with controller 72 via a cable, cord, or wireless communication. Mattress angle sensors 92f are most commonly used to measure the angular extent to which a patient is being laterally turned or tilted by an inflatable side bladder (or plurality of side bladders) that are used to assist in rotating that patient. That is, angle sensors 92f measure how far a patient is being rolled in a direction toward a side of support deck 30 (rather than toward head end 38 or foot end 40).

Mattress pressure sensors 92g are adapted to determine an inflation pressure of one or more bladders inside of the mattress 42. As with mattress angle sensors 92f, pressure sensors 92g are typically positioned inside of mattress 42 and in communication with controller 72 via a cable, cord, or a wireless communication link. Although a wide variety of mattress angle sensors 92f and/or pressure sensors 92g may be used, in some embodiments, sensors 92f and 92g are the same as the angle and pressure sensors disclosed in commonly assigned U.S. Pat. No. 9,468,307 issued to Lafleche et al. and entitled INFLATABLE MATTRESS AND CONTROL METHODS, and/or U.S. Pat. No. 9,526,349 issued to Lafleche et al. and entitled PATIENT SUPPORT COVER, and/or any of the patent references incorporated into either of these patents by reference. The complete disclosures of the U.S. Pat. Nos. 9,468,307 and 9,526,349 patents are incorporated herein by reference.

Deck width sensors 92h are adapted to determine the current width of support deck 30 in those embodiments of patient support apparatus 20 that are equipped with a variable width deck 30. Deck width sensors 92h may take on any of the forms disclosed in commonly assigned U.S. patent application Ser. No. 62/994,540 filed Mar. 25, 2020, by inventors Jason Connell et al. and entitled PATIENT SUPPORT WITH DECK WIDTH MONITORING AND CONTROL, the complete disclosure of which is incorporated herein by reference. Still other types of expandable width deck sensors may be used.

Deck length sensors 92i are adapted to determine the current length of support deck 30 in those embodiments of patient support apparatus 20 that are equipped with a variable length deck 30. Deck length sensors 92i may be implemented as the same type of sensor as any of the deck width sensors 92h, or they may take on other forms.

Room temperature sensor 92j may be any type of temperature sensor that is positioned onboard patient support apparatus 20 at a location that is exposed to the ambient air of the room in which patient support apparatus 20 is located. Similarly, room humidity sensor 92k, room sound sensor 92l, room air flow sensor 92m, and room light sensor 92n are any type of sensors that are capable of detecting humidity, sound, air flow, and ambient light, respectively, and that are positioned onboard patient support apparatus 20 at a suitable location for detecting these parameters.

A/C power sensor 92o is adapted to detect whether patient support apparatus 20 is currently plugged into an electrical outlet and receiving power from the electrical outlet (as opposed to operating on battery power).

Battery charge sensor 92p is adapted to detect a current charge level of any batteries that are positioned onboard patient support apparatus 20. In some embodiments, patient support apparatus 20 includes multiple batteries, and in such embodiments, patient support apparatus 20 includes multiple battery charge sensors 92p that detect the current charge level of each battery. In other embodiments, patient support apparatus 20 may not include any batteries, in which case battery charge sensor 92p is omitted.

Exit detection system 88 (FIG. 3) is configured to determine a weight of a patient positioned on support deck 30 and determine when the patient is moving and is likely to exit patient support apparatus 20. The particular structural details of exit detection system 88 can vary widely. In some embodiments, exit detection system 88 includes a plurality of force sensors 92q, such as, but not limited to, load cells that are arranged to detect the weight exerted on litter frame 28. By summing the outputs from each of the force sensors 92q, the total weight of the patient is determined (after subtracting the tare weight). Further, by using the known position of each of the force sensors 92q, controller 72 determines a center of gravity of the patient and monitors the center of gravity for movement beyond one or more thresholds. One method of computing the patient's center of gravity from the output of such force sensors is described in more detail in commonly assigned U.S. Pat. No. 5,276,432 issued to Travis and entitled PATIENT EXIT DETECTION MECHANISM FOR HOSPITAL BED, the complete disclosure of which is incorporated herein by reference. Other methods for determining a patient's weight and/or the weight of non-patient objects supported on litter frame 28 are disclosed in commonly assigned U.S. patent application Ser. No. 14/776,842, filed Sep. 15, 2015, by inventors Michael Hayes et al. and entitled PATIENT SUPPORT APPARATUS WITH PATIENT INFORMATION SENSORS, and commonly assigned U.S. patent application Ser. No. 14/873,734 filed Oct. 2, 2015, by inventors Marko Kostic et al. and entitled PATIENT SUPPORT APPARATUSES WITH MOTION MONITORING, the complete disclosures of both of which are incorporated herein by reference. Other systems for determining a patient's weight and/or detecting a patient's exit from patient support apparatus 20 may alternatively be used.

It will be understood that the aforementioned set of sensors 92a-q may be varied in different embodiments of patient support apparatus 20. That is, in some embodiments of patient support apparatus 20, one or more of sensors 92a-q may be omitted, one or more additional sensors may be added, and/or a combination of omitted and additional sensors may be included. Still further, in some embodiments, one or more meta-sensors may be included that detect one or more conditions of one or more sensors 92a-q themselves. Examples of suites of meta-sensors that are used to detect the condition of one or more other sensors onboard a patient support apparatus are disclosed in commonly assigned U.S. patent application Ser. No. 16/367,872 filed Mar. 28, 2019 by inventors Marko Kostic et al. and entitled PATIENT SUPPORT APPARATUSES WITH MULTI-SENSOR FUSION, the complete disclosure of which is incorporated herein by reference. Any of the embodiments of patient support apparatus 20 disclosed herein may include one of more of the meta-sensors disclosed in the aforementioned '872 application, and/or one or more other types of meta-sensors.

In addition to the aforementioned sensors, patient support apparatus 20 may further be adapted to sense any of the characteristics described in the patent applications identified in the chart below, as well as to include any of the sensors disclosed in these patent applications:

| Patent/App. | Filing Date | Title | Characteristic Detected |
|---|---|---|---|
| 5,276,432 | Jan. 15, 1992 | Patient Exit Detection Mechanism | Patient's location (center of gravity) |
| | | for Hospital Bed | |
| 7,699,784 | Jul. 5, 2007 | System for Detecting and | Patient's heart rate, breathing rate, and |
| | | Monitoring Vital Signs | other vital signs |
| 9,320,444 | Mar. 14, 2014 | Patient Support Apparatus with | Patient sleep quantity, quality, and |
| | | Patient Information Sensors | other sleep parameters; patient weight |
| 61/449,182 | Mar. 4, 2011 | Sensing System for Patient | Patient interface pressures, vital signs, |
| | | Supports | |
| 14/692,871 | Apr. 22, 2015 | Person Support Apparatus with | Patient movement |
| | | Position Monitoring | |
| 14/873,734 | Oct. 2, 2015 | Person Support Apparatus with | Patient and object weights, movement, |
| | | Motion Monitoring | and position |
| 14/928,513 | Oct. 30, 2015 | Person Support Apparatus with | A patient's activity, time out of bed, |
| | | Patient Mobility Monitoring | number of steps, and other activity data |
| 14/578,630 | Dec. 22, 2014 | Video Monitoring System | Patient turns, bed sore assessment |
| | | | scores, eating and sleeping, exit |
| | | | detection system status, etc. |
| 15/346,779 | Nov. 9, 2016 | Person Support Apparatus with | Patient vital signs, position, movement |
| | | Acceleration Detection | |
| 15/809,351 | Nov. 10, 2017 | Patient Support Apparatuses with | Patient mobility score and/or |
| | | Mobility Assessment | assessments |
| 15/709,586 | Sep. 20, 2017 | Systems and Methods for | Cleanliness and/or usability status of a |
| | | Determining the Usability of | patient support apparatus |
| | | Person Support Apparatuses | |

Each of these commonly assigned patent applications is incorporated herein by reference in their entirety.

Patient support apparatus 20 may also include one or more patient presence/movement detectors that are adapted to automatically detect whether or not a patient is currently present on patient support apparatus 20, as well as, in some instances, to detect movement and/or the position of the patient when the patient is supported on patient support apparatus 20. The specific components of patient the presence detector and/or manner in which it detects a patient's presence/absence/movement/location may vary from embodiment to embodiment. In one embodiment, the patient presence detector uses force sensors 92q. In another embodiment, the patient presence detector may alternatively be implemented using one or more thermal sensors mounted to patient support apparatus 20 that detect the absence/presence of the patient and/or the position of the patient's head on patient support apparatus 20. Further details of such a thermal sensing system are disclosed in commonly assigned U.S. patent application Ser. No. 14/692,871 filed Apr. 22, 2015, by inventors Marko Kostic et al. and entitled PERSON SUPPORT APPARATUS WITH POSITION MONITORING, the complete disclosure of which is incorporated herein by reference. In still other embodiments, the patient presence detector detects the absence/presence/movement/location of a patient using one or more of the methods disclosed in commonly assigned U.S. patent application Ser. No. 14/928,513 filed Oct. 30, 2015, by inventors Richard Derenne et al. and entitled PERSON SUPPORT APPARATUSES WITH PATIENT MOBILITY MONITORING, the complete disclosure of which is also hereby incorporated herein by reference. In yet other embodiments, the patient presence detector includes one or more video cameras for detecting the patient's presence, absence, movement, and/or position, such as disclosed in commonly assigned U.S. patent application Ser. No. 14/578,630 filed Dec. 22, 2014, by inventors Richard Derenne et al. and entitled VIDEO MONITORING SYSTEM, the complete disclosure of which is also hereby incorporated herein by reference. In yet another alternative embodiment, the presence, absence, movement and/or position of a patient is detected using a pressure sensing mat. The pressure sensing mat is positioned on top of the mattress or support deck 30, such as is disclosed in commonly assigned U.S. patent application Ser. No. 14/003,157 filed Mar. 2, 2012, by inventors Joshua Mix et al. and entitled SENSING SYSTEM FOR PATIENT SUPPORTS, the complete disclosure of which is also incorporated herein by reference. In still other embodiments, the patient presence detector may take on still other forms.

Mattress 42 may be a powered mattress that includes one or more inflatable chambers that are inflated under the control of controller 72 of patient support apparatus 20. In some embodiments, mattress 42 is configured to apply one or more therapies to a patient (e.g. a percussion therapy) and/or to assist in automatically turning a patient. Still further, in some embodiments, mattress 42 may include one or more vital sign sensors built into it that detect one or more of the patient's vital signs, as well as one or more pressure sensors that detect fluid pressure inside of the chambers and/or interface pressure between the patient and the mattress. An illustrative mattress 152 suitable for use with patient support apparatus 20 that includes many of these features is disclosed in commonly assigned U.S. patent application Ser. No. 13/836,813 filed Mar. 15, 2013, by inventors Patrick Lafleche et al. and entitled INFLATABLE MATTRESS AND CONTROL METHODS, the complete disclosure of which is incorporated herein by reference.

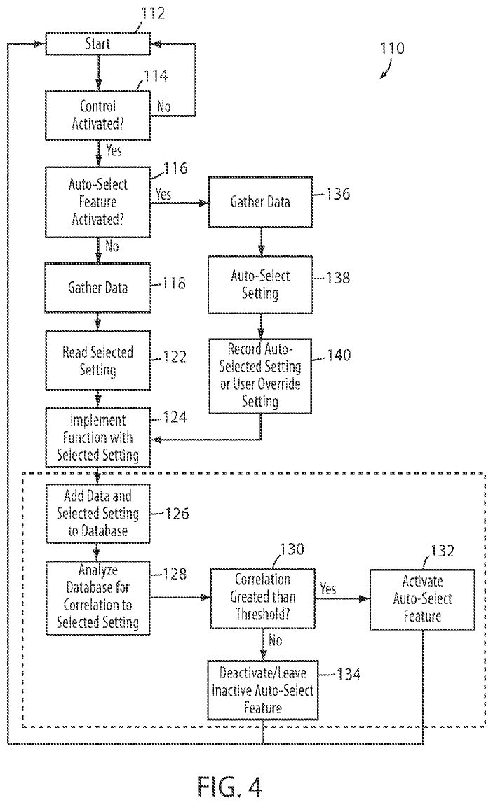

In some embodiments of patient support apparatus 20, controller 72 is adapted to perform an automatic setting selection algorithm 110 in response to a caregiver or other user activating one or more controls on patient support apparatus. FIG. 4 illustrates in greater detail one embodiment of automatic setting selection algorithm 110. Algorithm 110 begins at a step 112 where it proceeds to step 114. At step 114, controller 72 determines if any control 72 that is to be monitored as part of algorithm 110 has been activated by the user. The particular controls 72 that are monitored at step 114 may vary from patient support apparatus 20. In general, the controls 72 that may be monitored at step 114 are any controls that perform any function on patient support apparatus 20 wherein the function involves at least two different settings that the user can choose from. Depending upon which setting the user chooses, the function is carried out in a different manner. For example, one control 72 that may be monitored as part of step 114 is the exit detection control 52a (FIG. 2). Other controls include, but are not limited to, any of navigation controls 52b-f. Still other controls 72 that may be monitored at step 114 of algorithm 110 are discussed below.

If the user activates a control 52 at step 114, controller 72 proceeds to step 116 (FIG. 4). If the user does not activate a control at step 114, controller 72 proceeds back to step 112, and continues to wait until a control is activated at step 114. At step 116, controller 72 determines if an auto-select feature has been activated or not. The auto-select feature is described in more detail below. In general, the auto-select feature is activated when sufficient data has been gathered from past activations of a particular control 52 at step 114 to enable controller 72 to accurately predict which setting the user will select when carrying out the function associated with that particular control 52. As will be discussed in greater detail below, this previously gathered data may be stored onboard patient support apparatus 20 (e.g. in memory 74), or it may be stored at a remote computer (e.g. remote computer 100), or it may be stored in multiple locations.

If the auto-select feature has not been activated when controller 72 performs step 114, controller 72 proceeds to step 118 (FIG. 4). At step 118, controller 72 takes readings from a plurality of sensors, including, but not limited to, any one or more of sensors 92a-q. In addition to taking readings from a plurality of sensors onboard patient support apparatus 20, controller 72 may gather additional data from additional sensors and/or computers/servers that are positioned offboard patient support apparatus 20. For example, controller 72 may determine at step 118 the location of patient support apparatus 20 within the healthcare facility (e.g. room number, bay identifier, ward, and/or treatment unit). Such location data may be gathered in a variety of different manners, such as, but not limited to, communicating with a fixed headwall unit located adjacent to the patient support apparatus. Alternatively, or additionally, controller 72 may utilize network transceiver 84 to communicate with one or more servers on local area network 68 that include the current location of patient support apparatus 20. Still other manners of determining the location of patient support apparatus 20 may be used. In some embodiments, any of the manners of determining the location of a patient support apparatus may be used that are disclosed in commonly assigned U.S. patent application Ser. No. 62/868,947 filed Jun. 20, 2019, by inventors Thomas Durlach et al. and entitled CAREGVER ASSISTANCE SYSTEM, the complete disclosure of which is incorporated herein by reference.

In addition to gathering location data, controller 72 may also and/or alternatively gather data about the patient assigned to patient support apparatus 20 at step 118 (FIG. 4). Such patient data may include data indicating what level of a fall risk the patient is, what risk level the patient is at for developing bed sores, what medical condition(s) the patient is suffering from, the age of the patient, the weight of the patient, historical data about use of the control activated at step 114 in the past for this particular patient, etc. This data may be gathered from sending an inquiry to EMR server 96, querying data stored in memory 74, and/or in other manners. In some embodiments, some or all of this data may be gathered through communication of patient support apparatus 20 with a caregiver assistance system of the type disclosed in the commonly assigned 62/868,947 application filed Jun. 20, 2019, and previously incorporated herein by reference.

In addition to patient data, data regarding the caregiver associated with the patient assigned to patient support apparatus 20 may also be gathered at step 118. Such caregiver data may include an identification of the caregiver, rounding data about the rounds performing by the caregiver, as well as other information about the caregiver (e.g. age, gender, number of years of experience, etc.). Such caregiver information may be retrieved in any of the aforementioned manners, such as by communicating with one or more servers on network 68, such as, but not limited to, a caregiver scheduling server 98 that identifies which caregivers are assigned to which patients (or rooms or patient support apparatuses 20) and what shifts (e.g. times) the caregivers are scheduled to be present within the healthcare facility. Still other data may be gathered at step 118 beyond the data specifically mentioned above.

After gathering data at step 118, controller 72 moves to step 122 where it determines what setting the user has selected for carrying out the function associated with the control that was activated at step 114. For example, if the control activated at step 114 is an exit detection system activation control (e.g. control 52 a), the caregiver has a choice of what sensitivity level to activate the exit detection system 88 at. The exit detection control screen 60 of FIG. 5 better illustrates this setting selection process. In some embodiments, controller 72 is configured to display control screen 60 in response to a caregiver pressing control 52a (and/or in response to other controls 52 that may be activated). Control screen 60 includes three sensitivity setting selectors 129a-c. If the caregiver activates (e.g. presses on) low sensitivity setting selector 120a, controller 72 arms exit detection system 88 with a low sensitivity—meaning that exit detection system 88 will only issue an exit alert if the patient makes a relatively large amount of movement toward exiting patient support apparatus 20. If the caregiver activates (e.g. presses on) medium sensitivity setting selector 120b, controller 72 arms exit detection system 88 with a medium sensitivity—meaning that exit detection system 88 will issue an exit alert if the patient makes a medium amount of movement toward exiting patient support apparatus 20 (e.g. less than the amount required for triggering an exit alert when low sensitivity setting selector 120a is selected). Finally, if the caregiver activates (e.g. presses on) high sensitivity selector 120c, controller 72 arms exit detection system 88 with a high sensitivity—meaning that exit detection system 88 will issue an exit alert if the patient makes even a relatively small amount of movement toward exiting patient support apparatus 20 (e.g. less than the amount of movement required for triggering an exit alert when either medium or high sensitivity setting selectors 120a or 120b are selected). Suitable manners of implementing these different sensitivity levels of exit detection system 88, as well as suitable manners for implementing exit detection system 88 itself, are described in more detail in commonly assigned U.S. patent application Ser. No. 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. Still other manners of implementing different sensitivity levels and/or exit detection system 88 itself may, of course, be used.

Continuing with the example of algorithm 110 as it applies to an exit detection arming control being activated at step 114, controller 72 determines at step 122 which sensitivity setting the caregiver selects or, if the caregiver does not make an active selection, which sensitivity setting exit defaults to using in the absence of an active selection. In other words, controller 72 determines at step 122 whether the exit detection system 88 will be armed with a low, medium, or high sensitivity.

At step 124 of algorithm 110 (FIG. 4), controller 72 implements the function activated at step 114 using the setting selected by the user (or by default) at step 122. Thus, in the exit detection system example, controller 72 arms exit detection system 88 at step 122 with whatever sensitivity level the caregiver has selected (low, medium, or high) at step 122, or in the case of no active selection, the default sensitivity level.

After arming exit detection system 88 at step 124, controller 72 either proceeds to carry out steps 126 through 134 itself, or it transmits (via transceiver 84) a set of data to remote computer 100 and remote computer 100 carries out steps 126 through 134. The set of data includes the data gathered at step 118, an identification of the control activated at step 114, and an identification of the setting selected at step 122. At step 126, either controller 72 or remote computer 100 adds the aforementioned set of data (gathered at steps 114, 118, and 122) to a database. The database contains similar sets of data that were gathered previously when a user activated that same control at step 114. For example, whenever control 52a is activated at step 114, the data gathered at step 114, 118 and 122 is added to the database. The database therefore contains readings of the data gathered at step 118 for each time a particular setting was selected at step 122. In other words, every time a caregiver previously chose a particular setting at step 122 (or it was chosen by default), the database contains a corresponding set of data that was gathered at step 118 for that particular setting selection.

At step 128, either controller 72 or remote computer 100 analyzes the data in the database to determine what level of correlation exists, if any, between the data gathered at step 118 and the particular selection made at step 122. This correlation analysis may be performed in a variety of different manners, including, but not limited to, using one or more machine learning algorithms, such as, but not limited to, supervised learning methods, unsupervised learning methods, semi-supervised learning methods, reinforcement learning methods, feature learning methods, and/or self-learning methods. The model used by the machine learning algorithm may be based upon any one or more of the following: an artificial neural network, a decision tree, a support vector machine, a regression analysis, a Bayesian network, and/or a training model. Regardless of which specific machine learning algorithm and/or model is utilized, controller 72 or remote computer 100 carry out the analysis at step 128 in order to find a reliable correlation between at least one of the items of data gathered at step 118 and the corresponding setting selected at step 122. That is, controller 72 or remote computer 100 look for at least one item of the data (and possibly a set of such data) that, over substantially all of the past times that the control of step 114 was activated, is a reliable indicator of which setting the user will select at step 122.

Returning to the exit detection system example, controller 72 or remote computer 100 is configured to look at all of the past times that exit detection control 52a was activated and see if any of the data gathered at step 118 for each of these corresponding activations of control 52 reliably correlates to the setting that was selected at step 122. Such analysis may determine, as an example, that a particular caregiver A always selects sensitivity level 120b, or that any patient support apparatus 20 positioned within a particular wing of the hospital (or on a particular ward or treatment unit) has sensitivity level 120a selected. Alternatively or additionally, the analysis may reveal that whenever caregiver A activates exit detection control 52a during the evening hours in a particular location of the healthcare facility, he or she chooses sensitivity level 120c. As yet another example, the analysis may reveal that if the patient is over a certain age and/or if the patient has a fall risk greater than a certain threshold, the caregiver activates sensitivity level 120c. Still other types of correlations between one or more of the data items gathered at step 118 and the selection made at step 122 may be discovered as part of the analysis carried out at step 128.

After carrying out the analysis at step 128, controller 72 or remote computer 100 compares the correlation(s), if any, determined at step 128 to a threshold at step 130 (FIG. 4). In some embodiments, the threshold is configurable by the user, such as by navigating to a setting screen, or the like, on display 54 that allows the user to change the threshold. The threshold determines how reliable the correlations need to be before controller 72 will switch to making an automatic selection in the future of which setting the user will select at step 122, thereby relieving the user of the need to make this manual selection in the future. In some instances, the threshold may be 100 percent, in which case the auto-selection feature will not be implemented unless a perfect correlation can be found between one or more of the data items gathered at step 118 and the particular setting selected at step 122. In other instances, the threshold may be less so that the auto-selection feature may be activated despite the fact that the data gathered at step 118 from past activations of the particular control (at step 114) may not allow for a completely reliable prediction to be made of which particular setting the caregiver makes at step 122.

If the correlation is determined at step 130 to be greater than the threshold (FIG. 4), controller 72 or remote computer 100 executes step 132. At step 132, controller 72 or remote computer 100 activates the setting auto-select feature. As noted, the setting auto-select feature is a feature in which controller 72 automatically makes the selection of a particular setting (normally performed at step 122) in response to the user activating a particular control at step 114. Thus, for example, if the auto-select feature has been activated and the caregiver presses on exit detection control 52a, controller 72 automatically chooses a sensitivity level 120a, b, or c for the caregiver so that the caregiver does not need to manually make this selection. This automatic selection is made based upon the analysis of the past history (performed at step 128) of the activations of exit detection control 52a and the particular settings 120a-c that were manually made by the caregiver for those past activations.

If remote computer 100 performs steps 126 through 134, it activates the auto-select feature at step 132 by sending a message to controller 72 (via transceiver 84) indicating that controller 72 should automatically select a setting in response to a user activating a particular control 52 (e.g. a caregiver activating control 52a) on patient support apparatus 20. In some embodiments, controller 72 makes this automatic selection itself, while in other embodiments, controller 72 may transmit the appropriate data to remote computer 100 and have remote computer 100 determine which automatic selection to make. If controller 72 performs steps 126 through 134, it activates the auto-select feature itself and does not need to transmit a message to remote computer 100, or receive a message from remote computer 100, in order activate the auto-select feature.

If controller 72 or remote computer 100 determines at step 130 that the correlation is less than the threshold, it proceeds to step 134 where it deactivates the auto-select feature (if previously deactivated), or leaves the auto-select feature inactive (if previously inactive). After completing step 134 (and 132), algorithm 110 returns to start step 112.

Returning to step 116 of algorithm 110 (FIG. 4), if the auto-select feature is active at the time controller 72 executes step 116, controller 72 proceeds to step 136 instead of step 118. At step 136, controller 72 gathers data. The data that is gathered at step 136 is, in some embodiments, the same data that is gathered at step 118. In other embodiments, the data gathered at step 136 may be only that portion of the data gathered at step 118 that has been determined (via the analysis at step 128) to be reliably predictive of what setting the user will choose. In either situations, after gathering the data at step 136, controller 72 proceeds to step 138. At step 138, controller 72 uses the data gathered at step 136 along with the model previously developed via the past analyses carried out at steps 128 to automatically predict what setting the caregiver will choose, and to automatically select that setting for the caregiver. In other words, controller 72 uses the machine learning algorithm of step 128 with the data gathered at step 136 to automatically select the setting for the caregiver at step 138. In the exit detection system example, controller 72 automatically selects one of sensitivity levels 120a, 120b, or 120c at step 138 in response to the caregiver activating control 52a (or another control that arms exit detection system 88).

In some embodiments, controller 72 carries out step 138 by plugging in the data from step 136 into the predictive model developed at step 138. In other embodiments, controller 72 sends the data gathered at step 136 (and an identification of the specific control 52 activated at step 114) to remote computer 100 and remote computer 100 analyzes this data to make the automatic selection of the corresponding setting. In the latter embodiment, remote computer 100 then sends a message back to patient support apparatus 20 instructing it what setting to select at step 138.

From step 138, controller 72 proceeds to step 140 where it determines whether the user will utilize the setting automatically selected at step 138 or override that automatically selected setting. Regardless of whether the user accepts the automatic setting selection or manually overrides it with a different setting, controller 72 records the selected setting (automatically made or manually overridden) and proceeds to step 124. At step 124, as described previously, controller 72 carries out the function associated with the control that was activated at step 114 using the setting that was either automatically selected at step 138 or manually overridden by the caregiver at step 140. From step 124, algorithm 110 proceeds to steps 126-134 in the manner previously described, and the setting automatically selected at step 128 (or overridden at step 140), as well as the data gathered at step 136 is added to the database at step 126 and becomes part of the corpus of data utilized by the machine learning algorithm executed by controller 72 or remote computer 100 at step 128.

It will be understood that when remote computer 100 is configured to carry out the analysis of step 128 (FIG. 4), it may be configured to utilize data added at step 126 from a plurality of patient support apparatuses 20 positioned within the same healthcare facility. Thus, when a user activates a control on a particular patient support apparatus 20, the data utilized in step 128 of the algorithm 110 performed for that particular patient support apparatus 20 may comprise data that was gathered by remote computer 100 from other patient support apparatuses 20. In still other embodiments, remote computer 100 may carry out the analysis of step 128 using data from other healthcare facilities. In such embodiments, remote computer 100 may be positioned outside of the healthcare facility (and accessible to network 68 via the Internet), or remote computer 100 may be in communication with another remote computer via the Internet that has access to data gathered from patient support apparatuses 20 positioned at other healthcare facilities.

Although algorithm 110 has been primarily described so far as applying to an auto-selection feature in response to a user activating exit detection control 52a, it will of course be understood that algorithm 110 may be applied to a large number of different controls on patient support apparatus 20. Several of these additional controls are described below with respect to FIGS. 6-10. Still other controls beyond those described with respect to FIG. 6-10 may be used with algorithm 110. Still further, it will be understood that a single patient support apparatus 20 may use algorithm 110 for one or more different controls positioned thereon, and the auto-select feature may, at a particular time, be turned on for certain controls 52 and turned off for certain other controls. Over time, the combination of controls for which the auto-select feature is turned on may vary. Still further, in some embodiments, patient support apparatus 20 is configurable by a user as to which controls 52 algorithm 110 is to be applied and which controls 52 algorithm 110 is not to be applied.

FIG. 6 illustrates a lockout control screen 90 that may be displayed on display 54 of patient support apparatus 20 in response to a user pressing on lockout control 52e (or another control 52 adapted to trigger the display of control screen 90). Control screen 90 allows a user to select which actuators on patient support apparatus 20 are to be temporarily locked out (i.e. the actuators are not driven in response to a user, such as a patient, pressing on a control that otherwise would cause the actuator to be driven). Controller 72, in some embodiments, applies algorithm 110 to the pressing of lockout control 52e. In such embodiments, if the user presses on lockout control 52e, controller 72 proceeds to either step 136 of algorithm 110 or step 118 of algorithm 110. In such embodiments, the settings referred to in algorithm 110 correspond to the specific set of lockouts that are to be activated and not activated by the user. These specific lockouts will now be described.

Lockout control screen 90 of FIG. 6 includes an HOB 30°+lockout setting selector 120d, a HOB lockout setting selector 120e, a thigh lockout setting selector 120f, and a low height lockout setting selector 120g. If the user activates HOB 30°+lockout setting selector 120d, Fowler lowering control 64d (FIG. 2) will not allow a user to lower Fowler section 44 below a specified angle (e.g. thirty degrees) with respect to horizontal (or with respect to a component of patient support apparatus 20 that is typically horizontally oriented, but which may be slightly off-horizontal if, for example, patient support apparatus 20 is not on a level floor). Thus, if a user presses on Fowler lowering control 64d when the Fowler section 44 is at, say a forty-five degree angle, controller 72 will drive Fowler actuator 78 lower until it reaches the limit of, in this case, thirty degrees. Once at thirty-degrees, controller 72 stops driving the Fowler section 44 any lower, regardless of whether or not a user keeps pressing on Fowler lowering control 64d.

If a user activates HOB lockout setting selector 120e (FIG. 6) controller 72 no longer moves Fowler section 44 upward in response to a user pressing on Fowler raise control 64c and no longer moves Fowler section 44 downward in response to a user pressing on Fowler lower control 64d. Thus, when HOB lockout setting selector 120e is activated, no movement of fowler section 44 occurs in response to a user pressing controls 64c and/or 64d.

If a user activates thigh lockout setting selector 120f (FIG. 6), controller 72 no longer activates gatch actuator 82 in response to a user pressing on either gatch raise motion control 64e or gatch lower motion control 64f. And if a user activates height lockout setting selector 120g, controller 72 no longer activates lift actuators 76 in response to a user pressing on either height raise motion control 64g or height lower motion control 64h.

Lockout control selectors 120d, 120e, 120f, and 120g are, in at least one embodiments, toggle selectors such that they alternate between activating and deactivating their respective lockouts as they are repeatedly pressed. Thus, if a user presses, for example, HOB lockout setting selector 120e to activate it, and then later decides to deactivate the HOB lockout, he or she simply presses setting selector 120e again.

As noted previously, algorithm 110 (FIG. 4) may be implemented with respect to lockout control screen 90. In such implementations, the user pressing on one or more controls, such as lockout control 52e (FIG. 2), may be monitored at step 114 of algorithm 110. In such embodiments, controller 72 then proceeds from step 114 to step 116 and determines if the auto-select feature has been activated or not. In this particular example, the auto-select feature refers to an automatic selection of one or more of lockout setting selectors 120d-g. If the auto-select feature has been activated, controller 72 proceeds to steps 136, 138, and 140, where it gathers data and then automatically activates one or more of the lockouts controlled by selectors 120d-g, thereby relieving the user of having to manually press on the corresponding selectors 120d-g. As a result, when the auto-select feature is activated, the mere act of a user pressing on control 52e causes controller 72 to automatically select which lockouts to activate without requiring the user to press any of selectors 120d-g (unless he or she wishes to override this automatic selection).

If the auto-select feature is not activated at step 116 of algorithm 110, as applied to lockout control screen 90, controller 72 proceeds through steps 118 and 122-134 in the manner previously described. The data gathered at step 118 (and step 136) may be the same as, or it may be different from, the data gathered at these steps when algorithm 110 is implemented with respect to automatic selection of a sensitivity level for the exit detection system 88, as described previously with respect to FIG. 5. Thus, controller 72 may gather data at steps 118 and 136 that relates to any one or more of the particular caregiver, location, patient, time of day, data from any of sensors 92a-p, exit detection system 88, etc. and use that data in the analysis step 128 and/or in the automatic selection step 140. The result will be that, after the database has been sufficiently populated, controller 72 automatically makes the desired selections of selectors 120d-g in response to a user activating control 52e, thereby relieving the caregiver of having to make such selections.

It will be understood that the database analyzed at step 128 of algorithm 110 when algorithm 110 is applied to lockout setting selection will be a different database than the database used at step 128 of algorithm 110 when algorithm 110 is applied to a different automatic selection function (e.g. the automatic exit detection sensitivity selection).

FIG. 7 illustrates a monitoring control screen 70 that may be displayed on display 54 of patient support apparatus 20 in response to a user pressing on monitoring control 52b (or another control 52 adapted to trigger the display of control screen 70). Control screen 70 allows a user to select which conditions of patient support apparatus 20 are to be monitored. Such monitoring involves issuing an alert, reminder, or other type of indication to the user whenever one or more of the monitored conditions deviates from its desired state. Controller 72, in some embodiments, applies algorithm 110 to the pressing of monitoring control 52b. In such embodiments, if the user presses on monitoring control 52b, controller 72 proceeds to either step 136 of algorithm 110 or step 118 of algorithm 110. In such embodiments, the settings referred to in algorithm 110 correspond to the specific set of conditions that are to be monitored and not monitored by the user. If the auto-select feature of algorithm 110 has been activated, then controller 72 automatically makes a selection of which conditions to monitor and which conditions not to monitor in response to the user pressing on monitoring control 52b. The specific conditions that can be monitored will now be described.

Monitoring control screen 70 includes a low height monitoring setting selector 120h, a right head end siderail monitoring setting selector 120i, a right foot end siderail monitoring setting selector 120j, a left head end siderail monitoring setting selector 120k, a left foot end siderail monitoring setting selector 120l, a HOB monitoring setting selector 120m, and a flat monitoring setting selector 120n. As with selectors 120d-g, selectors 12h-n are toggle selectors that, when sequentially pressed, alternate between activating and deactivating their corresponding monitoring functions. When low height monitoring setting selector 120h is activated, controller 72 monitors the height of litter frame 28 with respect to the ground and issues an alert if litter frame 28 is not at its lowest height (or, in some embodiments, exceeds a user-configurable height). When right head end siderail monitoring selector 120i is activated, controller 72 monitors the position of the right head end siderail 36 and, if it is lowered, issues an alert. Similarly, when any of right foot end, left head end, and/or left foot end siderail monitoring selectors 120j-1 are activated, controller 72 monitors the corresponding siderail 36 and, if it is lowered, issues an alert. When HOB monitoring setting selector 120m is activated, controller 72 monitors the angle of head section 44 with respect to horizontal (or generally horizontal) and issues an alert if that angle drops below thirty-degrees (or, in some embodiments, an angle that is user-configurable). When flat monitoring setting selector 120n is activated, controller 72 monitors the orientation of each of the pivotable sections of support deck 30 and issues an alert if any of the deck sections 44, 46, and/or 48 are pivoted to a non-horizontal orientations. Alternatively, or additionally, controller 72 may monitor the orientation of litter frame 28 when flat monitoring setting selector 120n is activated and issue an alert if litter frame 28 is moved out of a horizontal orientation.

In some embodiments, algorithm 110 (FIG. 4) is implemented with respect to monitoring control screen 70. In such implementations, the user pressing on one or more controls, such as monitoring control 52b (FIG. 2), is monitored at step 114 of algorithm 110. In such embodiments, controller 72 then proceeds from step 114 to step 116 and determines if the auto-select feature has been activated or not. In this particular example, the auto-select feature refers to an automatic selection of which one or more conditions of patient support apparatus 20 are to be monitoring by the onboard monitoring system (i.e. which ones of monitoring setting selectors 120h-n are to be activated). If the auto-select feature has been activated, controller 72 proceeds to steps 136, 138, and 140, where it gathers data and then automatically activates the monitoring of the conditions associated with the activated selectors 120h-n, thereby relieving the user of having to manually press on the corresponding selectors 120h-n. As a result, when the auto-select feature is activated, the mere act of a user pressing on control 52b causes controller 72 to automatically begin monitoring the conditions that controller 72 deems to be most likely to be desired by the user, as determined from its previous analysis carried out at step 128. The user is, as noted, free to override or modify this automatic selection by pressing on any of selectors 120h-n.

If the auto-select feature is not activated at step 116 of algorithm 110, as applied to monitoring control screen 70, controller 72 proceeds through steps 118 and 122-134 in the manner previously described. The data gathered at step 118 (and step 136) may be the same as, or it may be different from, the data gathered at these steps when algorithm 110 is implemented with respect to the automatic selection of a sensitivity level for the exit detection system 88, and/or the automatic selection of a lockout, as described previously with respect to FIGS. 5 and 6. Thus, controller 72 may gather data at steps 118 and 136 that relates to any one or more of the particular caregiver, location, patient, time of day, or outputs from any of sensors 92a—and/or exit detection system 88, and/or other data, and use that data in the analysis step 128 and/or in the automatic selection step 140. The result will be that, after the database has been sufficiently populated, controller 72 automatically makes the desired selections of selectors 120h-n in response to a user activating control 52b, thereby relieving the caregiver of having to make such selections.

It will be understood that the database analyzed at step 128 of algorithm 110 when algorithm 110 is applied to the monitoring setting selection will be a different database than the database used at step 128 of algorithm 110 when algorithm 110 is applied to other automatic selection functions (e.g. the automatic exit detection sensitivity selection, the automatic selection of a lockout, and/or the automatic selection of other features discussed herein).

FIG. 8 illustrates a mattress therapy control screen 144 that may be displayed on display 54 of patient support apparatus 20 in response to a user pressing on a mattress control (not shown) adapted to trigger the display of screen 144. In some embodiments, the mattress control is an icon that is displayed on display 54 in response to a user navigating to a particular screen, such as a screen that is displayed after a user has pressed on control 52f. Regardless of the particular control used to navigate to it, mattress therapy control screen 144 allows a user to select different settings for a particular patient therapy that is to be applied to the patient using mattress 42. In this particular example, the therapy is a lateral rotation therapy, although it will be understood by those skilled in the art that other types of mattress therapies may be controlled via a mattress therapy control screen, and that algorithm 110 may be applied to such other types of mattress therapies.

The lateral rotation therapy involves inflating a first side of the mattress 42 to such an extent that the patient is rotated toward his or her opposite side by a user-configurable amount. After that rotation has been accomplished for a user-configurable amount of time, that side of the mattress is deflated and the user returns to a generally flat orientation for a user-configurable amount of time. After that, the opposite side of the mattress 42 is inflated to a user-configurable amount and held in that position for a user-configurable amount of time. This process is then repeated for as many times as the user designates. More specifically, the degree of rotation of the patient, the hold times at each position, and the length of the overall lateral therapy are all configurable by the user by pressing on setting selectors 1200-y.

Controller 72, in some embodiments, applies algorithm 110 to the pressing of the mattress control used to trigger the display of mattress control screen 144. In such embodiments, if the user presses on that control, controller 72 proceeds to either step 136 of algorithm 110 or step 118 of algorithm 110. In such embodiments, the settings referred to in algorithm 110 correspond to the specific settings for the mattress therapy that are selectable by the user via selectors 1200-y. If the auto-select feature of algorithm 110 has been activated, then controller 72 automatically makes a selection of these mattress therapy settings. The specific settings will now be described.

Mattress therapy control screen 144 includes a left zero hold time setting selector 120o, a left five minute hold time setting selector 120p, a left ten minute hold time setting selector 120q, a left angle setting selector 120r, a zero flat hold time selector 120s, a five minute flat hold time selector 120t, a ten minute flat hold time selector 120u, a right zero hold time setting selector 120v, a right five minute hold time setting selector 120w, a right ten minute hold time setting selector 120x, and a right angle setting selector 120y. When a user activates one of the left setting selectors 120o, 120p, or 120q, controller 72 is adapted to hold the patient in the leftward rotated position for the amount of time corresponding to the activated selectors 1200-q when performing lateral rotation therapy. When the user slides angle setting selector 120r to different positions along the arcuate line 142a shown in FIG. 8, the user is able to select the degree of angular rotation of the patient when the patient is rotated to his or her left side. Thus, depending upon what angle the user selects via selector 120r, controller 72 inflates the inflatable bladder(s) on the right side of mattress 42 to a different amounts in order to effectuate the desired amount of patient rotation.

When the user selects one of selectors 120s-v, controller 72 retains the patient in a flat (i.e. non-rotated) position for the amount of time corresponding to the particular selector 120s-v that was activated by the user. When the user activates one of the right setting selectors 120v, 120w, or 120x, controller 72 is adapted to hold the patient in the rightward rotated position for the amount of time corresponding to the activated selectors 120v-x when performing lateral rotation therapy. When the user slides angle setting selector 120y to different positions along the arcuate line 142b shown in FIG. 8, the user is able to select the degree of angular rotation of the patient when the patient is rotated to his or her right side. Thus, depending upon what angle the user selects via selector 120y, controller 72 inflates the inflatable bladder(s) on the left side of mattress 42 to a different amounts in order to effectuate the desired amount of patient rotation.

When carrying out the lateral rotation therapy of FIG. 8, the user selects only a single one of the three left rotational selectors 1200-q, only a single one of the three flat selectors 120s-v, and only a single one of the three right rotational selectors 120v-x.

In those embodiments where algorithm 110 (FIG. 4) is implemented with respect to mattress therapy control screen 144, controller 72 monitors the user pressing of one or more controls that trigger the display of screen 144. When such pressing is detected, controller 72 then proceeds from step 114 to step 116 and determines if the auto-select feature has been activated or not. In this particular example, the auto-select feature refers to an automatic selection of the times of rotation for the left, right, and flat orientations, as well as the angular degrees of right and leftward rotations. If the auto-select feature has been activated, controller 72 proceeds to steps 136, 138, and 140, where it gathers data and then automatically activates the lateral rotation settings associated with the activated selectors 1200-y, thereby relieving the user of having to manually press on the corresponding selectors 1200-y. As a result, when the auto-select feature is activated, the mere act of a user pressing on a mattress control causes controller 72 to automatically implement settings for carrying out lateral rotation therapy that controller 72 deems to be most likely to be desired by the user, as determined from its previous analysis carried out at step 128. The user is, as noted, free to override or modify this automatic selection by pressing on any of selectors 1200-y.

If the auto-select feature is not activated at step 116 of algorithm 110, as applied to mattress therapy control screen 144, controller 72 proceeds through steps 118 and 122-134 in the manner previously described. The data gathered at step 118 (and step 136) may be the same as, or it may be different from, the data gathered at these steps when algorithm 110 is implemented with respect to other functions (e.g. the automatic selection of a sensitivity level for the exit detection system 88, the automatic selection of a lockout, etc.). Thus, controller 72 may gather data at steps 118 and 136 that relates to any one or more of the particular caregiver, location, patient, time of day, or outputs from any of sensors 92a—and/or exit detection system 88, and/or other data, and use that data in the analysis step 128 and/or in the automatic selection step 138. The result will be that, after the database has been sufficiently populated, controller 72 automatically makes the desired selections of selectors 1200-y in response to a user activating a mattress therapy control, thereby relieving the caregiver of having to make such selections.

It will be understood that the database analyzed at step 128 of algorithm 110 when algorithm 110 is applied to the mattress therapy control selection will be a different database than the database used at step 128 of algorithm 110 when algorithm 110 is applied to other automatic selection functions (e.g. the automatic exit detection sensitivity selection, the automatic selection of a lockout, and/or the automatic selection of other features discussed herein).

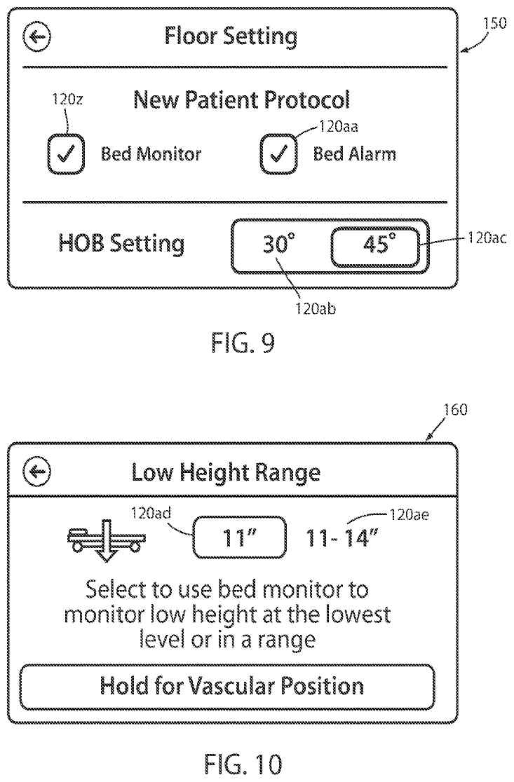

FIG. 9 illustrates a new patient protocol screen 150 that may be displayed on display 54 of patient support apparatus 20 in response to a user pressing on a new patient setting control (not shown) adapted to trigger the display of screen 150. In some embodiments, the new patient setting control is an icon that is displayed on display 54 in response to a user navigating to a particular screen, such as a screen that is displayed after a user has pressed on control 52f. Regardless of the particular control used to navigate to it, new patient protocol screen 150 allows a user to select different settings for the patient support apparatus 20 that are to be applied by controller 72 whenever a new patient is assigned to that patient support apparatus 20. In this particular example, there are three such settings: a bed monitoring setting 120z, a bed alarm settings 120aa, a thirty-degree HOB angle setting 120ab, and a forty-five degree HOB setting selector 120ac. It will, of course, be understood by those skilled in the art that other types of new patient settings may be controlled via new patient protocol screen 150, and that algorithm 110 may be applied to such other types of new patient protocols.

The new patient protocol screen 150 allows a user of patient support apparatus 20 to define one or more functions of patient support apparatus 20 that are to be automatically implemented by controller 72 of patient support apparatus 20 for each new patient. Such settings can be manually overridden. However, new patient protocol screen 150 allows the user to define what functions are implemented in the absence of such a manual override.

Controller 72, in some embodiments, applies algorithm 110 to the pressing of the new patient protocol control used to trigger the display of new patient protocol screen 150. In such embodiments, if the user presses on that control, controller 72 proceeds to either step 136 of algorithm 110 or step 118 of algorithm 110. In such embodiments, the settings referred to in algorithm 110 correspond to the specific settings for the new patient protocol(s) that are selectable by the user via selectors 120z-ab. If the auto-select feature of algorithm 110 has been activated, then controller 72 automatically makes a selection of these new patient protocol settings.

New patient protocol screen 150 includes a bed monitoring setting selector 120z, a bed alarm setting selector 120aa, a thirty-degree HOB setting selector 120ab, and a forty-five degree HOB setting selector 120ac. When bed monitoring setting 120z is activated, controller 72 issues a reminder, alert, and/or other audio and/or visual indication to the user if a patient is present on patient support apparatus 20 and the monitoring system onboard patient support apparatus 20 has not been armed. The monitoring system is the same monitoring system discussed above with respect to control 52b. Thus, when setting 120z is activated, controller 72 automatically issues a reminder to the caregiver when a patient is present on support deck 30 and the monitoring system has not been armed. In some embodiments, the reminder is carried out in one or more of the manners disclosed in commonly assigned PCT patent application serial number PCT/US20/38462 filed Jun. 18, 2020, and entitled PATIENT SUPPORT APPARATUS WITH CAREGIVER REMINDERS, the complete disclosure of which is incorporated herein by reference. Other types of reminders can, of course, be implemented. When setting 120z is not activated, controller 72 does not issue a reminder to activate the onboard monitoring system, even if a patient is present onboard support deck 30.

The presence/absence of a patient onboard support deck 30 may be determined in any of the manners disclosed in commonly assigned PCT patent application serial number PCT/US2020/039587 filed Jun. 25, 2020, and entitled CAREGIVER ASSISTANCE SYSTEM, the complete disclosure of which is incorporated herein by reference (including references incorporated by reference into the aforementioned PCT/US2020/039587 application).

When bed alarm setting 120aa is activated, controller 72 issues a reminder, alert, and/or other audio and/or visual indication to the user if a patient is present on patient support apparatus 20 and exit detection system 88 has not been armed. In some embodiments, the reminder is carried out in one or more of the manners disclosed in commonly assigned PCT patent application serial number PCT/US20/38462 filed Jun. 18, 2020, and entitled PATIENT SUPPORT APPARATUS WITH CAREGIVER REMINDERS, the complete disclosure of which is incorporated herein by reference. Other types of reminders can, of course, be implemented. When setting 120aa is not activated, controller 72 does not issue a reminder to activate exit detection system 88, even if a patient is present onboard support deck 30.

When thirty-degree HOB setting selector 120ab is activated, controller 72 issues an alert whenever the HOB monitoring setting selector 120m (FIG. 7) is activated and the angle of Fowler section 44 is moved to less than thirty degrees (with respect to horizontal or with respect to a typically horizontal component of patient support apparatus 20). When forty-five degree HOB setting selector 120ac is activated, controller 72 issues an alert whenever the HOB monitoring setting selector 120m (FIG. 7) is activated and the angle of Fowler section 44 is moved to less forty-five degrees (with respect to horizontal or with respect to a typically horizontal component of patient support apparatus 20). Setting selectors 120ab and 120ac therefore allow the user to configure what angular orientation the Fowler section 44 should be maintained at (or above) before patient support apparatus 20 issues an alert to the caregiver (when the angular orientation of Fowler section 44 is being monitored).

Setting selectors 120z and 120aa are toggle selectors that alternate between activating and deactivating their corresponding setting when they are repeatedly pressed. Setting selectors 120ab and 120ac automatically cancel the other one out when they are selected. In other words, if setting selector 120ab is activated and the user presses on selector 120ac, selector 120ac is automatically activated and selector 120ab is automatically deactivated.

In those embodiments where algorithm 110 (FIG. 4) is implemented with respect to new patient protocol screen 150, controller 72 monitors the user pressing of one or more controls that trigger the display of screen 150. When such pressing is detected, controller 72 proceeds from step 114 to step 116 and determines if the auto-select feature has been activated or not. In this particular example, the auto-select feature refers to an automatic selection of the bed monitoring function, the bed alarm function, and the angular degree to which the Fowler section will trigger alerts. If the auto-select feature has been activated, controller 72 proceeds to steps 136, 138, and 140, where it gathers data and then automatically activates the functions associated with the activated selectors 120z-ac, thereby relieving the user of having to manually press on the corresponding selectors 120z-ac. As a result, when the auto-select feature is activated, the mere act of a user pressing on the control that brings up new patient protocol screen 150 causes controller 72 to automatically implement the settings that controller 72 deems to be most likely desired by the user, as determined from its previous analysis carried out at step 128. The user is, as noted, free to override or modify this automatic selection by pressing on any of selectors 120z-ac.

If the auto-select feature is not activated at step 116 of algorithm 110, as applied to new patient protocol screen 150, controller 72 proceeds through steps 118 and 122-134 in the manner previously described. The data gathered at step 118 (and step 136) may be the same as, or it may be different from, the data gathered at these steps when algorithm 110 is implemented with respect to other functions (e.g. the automatic selection of a sensitivity level for the exit detection system 88, the automatic selection of a lockout, etc.). Thus, controller 72 may gather data at steps 118 and 136 that relates to any one or more of the particular caregiver, location, patient, time of day, outputs from any of sensors 92a—and/or exit detection system 88, and/or other data, and use that data in the analysis step 128 and/or in the automatic selection step 140. The result will be that, after the database has been sufficiently populated, controller 72 automatically makes the desired selections of selectors 120z-ac in response to a user activating a new patient protocol control, thereby relieving the caregiver of having to make such selections.

It will be understood that the database analyzed at step 128 of algorithm 110 when algorithm 110 is applied to the new patient protocol setting selection will be a different database than the database used at step 128 of algorithm 110 when algorithm 110 is applied to other automatic selection functions (e.g. the automatic exit detection sensitivity selection, the automatic selection of a lockout, and/or the automatic selection of other features discussed herein).

FIG. 10 illustrates a low height selection screen 160 that may be displayed on display 54 of patient support apparatus 20 in response to a user pressing on a low height setting control (not shown) adapted to trigger the display of control screen 160. In some embodiments, the low height setting control is an icon that is displayed on display 54 in response to a user navigating to a particular screen, such as a screen that is displayed after a user has pressed on control 52f. Regardless of the particular control used to navigate to it, low height selection screen 160 allows a user to select which a height at which, when litter frame 28 is above it, controller 72 will issue an alert, and when litter frame 28 is at or below it, controller 72 will not issue an alert. In this particular example, the user is presented with two choices: an eleven inch height and a range of heights between eleven and fourteen inches.

Controller 72, in some embodiments, applies algorithm 110 to the pressing of the low height selection control used to trigger the display of low height selection screen 160. In such embodiments, if the user presses on that control, controller 72 proceeds to either step 136 of algorithm 110 or step 118 of algorithm 110. In such embodiments, the settings referred to in algorithm 110 correspond to the specific low height setting selected by the user via selectors 120ad or 120ae. If the auto-select feature of algorithm 110 has been activated, then controller 72 automatically makes a selection of the height above which controller 72 will issue an alert.

When a user selects eleven inch selector 120ad of height selection screen 160, controller 72 monitors the height of litter frame 28 when the monitoring system is armed and the user has activated the height monitoring setting selector 120h (FIG. 7) of that monitoring system. If the height of the litter frame goes above eleven inches after selectors 120ad and 120h have been activated, and the monitoring system is armed, controller 72 issues an alert and/or a reminder to the caregiver that the litter frame 28 has moved to an undesired height. When a user selects eleven-to-fourteen inch selector 120ae of height selection screen 160, controller 72 monitors the height of litter frame 28 when the monitoring system is armed and the user has activated the height monitoring setting selector 120h (FIG. 7) of that monitoring system. If the height of the litter frame goes above fourteen inches after selectors 120ae and 120h have been activated, and the monitoring system is armed, controller 72 issues an alert and/or a reminder to the caregiver that the litter frame 28 has moved to an undesired height. When controller 72 is configured to issue a reminder, the reminder may be carried out in one or more of the manners disclosed in commonly assigned PCT patent application serial number PCT/US20/38462 filed Jun. 18, 2020, and entitled PATIENT SUPPORT APPARATUS WITH CAREGIVER REMINDERS, the complete disclosure of which is incorporated herein by reference. Other types of reminders can, of course, be implemented. When setting 120h is not activated, controller 72 does not issue a reminder or alert when litter frame 28 moves to any height, regardless of whether selector 120ad or selector 120ae is activated or not.

Setting selectors 12ad and 120ae are toggle selectors that alternate between activating and deactivating each other when they are repeatedly pressed. In other words, if setting selector 120ad is activated and the user presses on selector 120ae, selector 120ae is automatically activated and selector 120ad is automatically deactivated.

In those embodiments where algorithm 110 (FIG. 4) is implemented with respect to low height selection screen 160, controller 72 monitors the user pressing of one or more controls that trigger the display of screen 160. When such pressing is detected, controller 72 proceeds from step 114 to step 116 and determines if the auto-select feature has been activated or not. In this particular example, the auto-select feature refers to an automatic selection of the height of litter frame 28 that will trigger an alert. If the auto-select feature has been activated, controller 72 proceeds to steps 136, 138, and 140, where it gathers data and then automatically selects the height that will trigger an alert (when litter frame 28 exceeds it), thereby relieving the user of having to manually press on a selectors 120ad or 120ae. As a result, when the auto-select feature is activated, the mere act of a user pressing on the control that brings up height selection screen 160 causes controller 72 to automatically select the threshold height controller 72 deems to be most likely desired by the user, as determined from its previous analysis carried out at step 128. The user is, as noted, free to override or modify this automatic selection by pressing on either of selectors 120ad-ae.

If the auto-select feature is not activated at step 116 of algorithm 110, as applied to low height selection screen 160, controller 72 proceeds through steps 118 and 122-134 in the manner previously described. The data gathered at step 118 (and step 136) may be the same as, or it may be different from, the data gathered at these steps when algorithm 110 is implemented with respect to other functions (e.g. the automatic selection of a sensitivity level for the exit detection system 88, the automatic selection of a lockout, etc.). Thus, controller 72 may gather data at steps 118 and 136 that relates to any one or more of the particular caregiver, location, patient, time of day, outputs from any of sensors 92a—and/or exit detection system 88, and/or other data, and use that data in the analysis step 128 and/or in the automatic selection step 140. The result will be that, after the database has been sufficiently populated, controller 72 automatically makes the desired selections of selectors 120ad-ae in response to a user activating a height selection control, thereby relieving the caregiver of having to make such selections.

It will be understood that the database analyzed at step 128 of algorithm 110 when algorithm 110 is applied to the low height selection function will be a different database than the database used at step 128 of algorithm 110 when algorithm 110 is applied to other automatic selection functions (e.g. the automatic exit detection sensitivity selection, the automatic selection of a lockout, and/or the automatic selection of other features discussed herein).

It will also be understood that the database analyzed at step 128 of algorithm 110—whether algorithm 110 is applied to any of the setting selections discussed above with respect to FIGS. 5-10, or to still other setting selections—will likely include data from more sensors (or other sources) that than what controller 72 deems is necessary for implementing the auto-selection feature. In other words, controller 72 will gather data at step 118 from a set of sensors (or other sources) that likely includes more data than is needed to accurately predict which setting should be selected at step 138. This additional data is analyzed at step 128, but controller 72 determines that it does not have sufficient predictive power to predict which setting the user prefers in response to the particular control activated at step 114. Accordingly, controller 72, after populating the database sufficiently to determine a correlation between the outputs of one or more sensors (and/or other sources) and the user's desired selection, no longer uses the outputs from all of the sensors (or other sources) read at step 118 (or 136) to predict the users selection. Stated still more simply, controller 72 gathers data from a relatively large set of sensors (and/or other sources) at step 118, but only uses the outputs of a subset of those sensors (and/or other sources) at step 138 when automatically selecting the user's preferred setting. As noted, this is because controller 72 searches for correlations between the entire set of data gathered at step 118 and the user's desired setting, but such correlations typically only exist for a subset of that data (and/or a subset of the sensors). Accordingly, the sensors (and/or other sources) whose outputs are not predictive of the user's desired setting are not used when automatically selecting the users preferred setting at step 138.

In some embodiments, the analysis carried out at steps 126 and 128 is the development of an algorithm for predicting what setting the user will select in response to the control selected at step 114. In such embodiments, if the analysis is carried out by remote computer 100, remote computer 100 transmits to patient support apparatus 20 a new algorithm after the database has been sufficiently populated with enough data for remote computer 100 to conclude that the new algorithm is sufficiently reliable for predicting the users setting selection. The transmission of the new algorithm represents the activation of the auto-selection feature such that, when controller 72 reaches step 116 and it has the new algorithm onboard, it proceeds to step 136 and 138 and uses the new algorithm to automatically select one or more settings for the user.

As mentioned above, the new algorithm may not utilize all of the outputs from the sensors that is collected at step 118. That is, either controller 72 (or remote computer 100) uses data from a relatively large set of sensors to determine if an algorithm can be formulated with sufficient predict power (done at steps 126-130). To the extent such an algorithm can be formulated, it typically will utilize only a subset of the data analyzed at steps 126-130. Accordingly, controller 72 (or remote computer 100) uses a broad set of information in its search for a sufficiently reliable algorithm and when that algorithm is found, it typically will only use a subset of that broad set of information when implementing the new algorithm for predicting and/or automatically selecting the user's preferred setting(s).

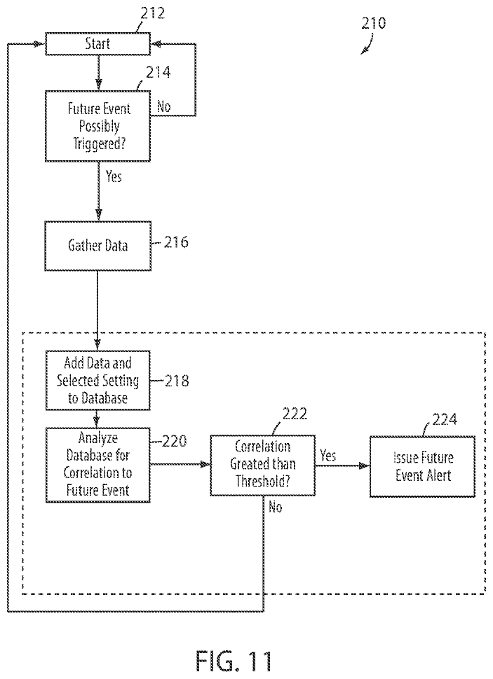

FIG. 11 illustrates a future event prediction algorithm 210 that controller 72 is adapted to execute in some embodiments of patient support apparatus 20. In some of those embodiments, controller 72 is adapted to execute both algorithm 110 and algorithm 210. While in other embodiments, controller 72 may be adapted to execute only a single one of algorithms 110 or 210. In still other embodiments, controller 72 may be adapted to execute still other artificial intelligence and/or machine learning algorithms for automatically selecting one or user-preferred settings and/or predicting one or more future events that are associated with patient support apparatus 20.

Algorithm 210 at a step 212 where it proceeds to step 214. At step 214, controller 72 determines if any action has taken place that will trigger it to begin monitoring for the possibility of a future event. Both the action that triggers the monitoring, as well as the future event, may vary from patient support apparatus 20. In general, the action that triggers the commencement of monitoring for a future event includes, but is not limited to, any one or more of the following: the presence of a patient on support deck 30; the commencement, termination, and/or continuation of a mattress therapy; the movement of patient support apparatus 20 to a different and/or particular location within the healthcare facility; the assignment of a bed sore risk score above a specified threshold to the patient associated with patient support apparatus 20; the assignment of a fall risk score above a specified threshold to the patient associated with patient support apparatus 20; the completion of a particular medical procedure; the movement (or lack of sufficient movement) of the patient over a time period; a diagnosis of the patient assigned to patient support apparatus 20; the arrival of a particular time of day and/or the arrival of a particular calendar day, week, or other time period; the determination and/or measurement of one or more patient characteristics (e.g. weight, age, height, vital signs, etc.) exceeding one or more threshold; combinations of any one or more of the foregoing/and/or still other patient-related and/or patient support apparatus-related actions.

The future event that is predicted by algorithm 210 may vary widely. Examples of the future event include, but are not limited to, the following: the development of one or more bed sores by the patient; the exiting of the patient from the patient support apparatus; the selection of one or more assessment scores by the patient (e.g. the Hospital Consumer Assessment of Healthcare Providers and Systems (HCAPHS) scores); the patient's use of the restroom; the development of Ventilator Associated Pneumonia (VAP) by the patient; the patient's use of a the nurse call system to contact a nurse; the need to change the patient's Foley bag; the need to zero the scale system onboard the patient support apparatus; the need to user a particular therapy with the patient (e.g. a mattress therapy, a Deep Vein Thrombosis (DVT) pump, a heel care boot, etc.); the need to take a patient's weight reading; the addition and/or removal of equipment on litter frame 28; the need to calibrate one or more items of patient support apparatus 20 (e.g. the scale and/or exit detection system 88); and/or other events.

The triggering action of step 214 may be sensed by controller 72 in a variety of different manners, depending upon the particular triggering action. In some embodiments, the triggering action is the entering of information into the patient support apparatus 20 via control panel 50, in which case controller 72 is directly fed the triggering information by the user. In other embodiments, the triggering action may be based on the output of one or more of sensors 92a-q (and/or other sensors), and in such cases, controller 72 reads this triggering information from the corresponding sensors. In other embodiments, the triggering action may be the receipt of information from an off-board source of information, such as remote computer 100, and/or another server or computer device that is in communication with local area network 68 (and thus patient support apparatus 20 via network transceiver 84). In still other instances, patient support apparatus 20 may receive information directly from another off-board source, such as a smart phone, portable computer, etc. that communicates directly (e.g. via Bluetooth) with a compatible transceiver onboard patient support apparatus 20. The particular information that is received from off-board the patient support apparatus 20 may vary. In some embodiments, it may include information from a patient's electronic medical record, information about a particular caregiver, information about a particular location of the healthcare facility, and/or other types of information.

For example, in at least some embodiments, controller 72 is triggered at step 214 when a patient is assigned to patient support apparatus 20 who may be at risk of developing a bed sore. In some of these embodiments, controller 72 may be triggered at step 214 by one or more sensors onboard patient support apparatus 20 that indicate the patient's presence onboard the patient support apparatus 20, as well as information from an electronic medical record of the patient's indicating the patient's risk score for developing bed sores (e.g. a Braden scale score). In other such embodiments, the triggering factor may just be the receipt of information by controller 72 indicating that the patient is at risk for developing bed sores, or that the patient has a specific bed sore risk score. In still others of such embodiments, one or more of the triggering factors may be different.

As another example, in at least some embodiments, controller 72 is triggered at step 214 when a patient is assigned to patient support apparatus 20 who may be at risk of falling. In some of these embodiments, controller 72 may be triggered at step 214 by one or more sensors onboard patient support apparatus 20 that indicate the patient's presence onboard the patient support apparatus 20, as well as information from an electronic medical record of the patient's indicating the patient's risk score for falling (e.g. a Morse fall risk score). In other such embodiments, the triggering factor may just be the receipt of information by controller 72 indicating that the patient is at risk for falling, that exit detection system 88 has been activated, or that the patient has a specific fall risk score. In still others of such embodiments, one or more of the triggering factors may be different.

If no triggering action is detected at step 214, controller 72 proceeds back to step 212, and continues to wait until a triggering action is detected at step 214. At step 216, controller 72 takes readings from a plurality of sensors, including, but not limited to, any one or more of sensors 92a-q. In addition to taking readings from a plurality of sensors onboard patient support apparatus 20, controller 72 may gather additional data, such as, but not necessarily limited to, any of the data that controller 72 gathers at step 118 of algorithm 110, as discussed above. Thus, for example, this additional data may patient data, location data, caregiver data, and/or other data.

The information gathered at step 216 also includes an indication of a “ground truth” about the future event that algorithm 210 is being used to predict. Thus, for example, if algorithm 210 is being used to predict the development of bed sores in a patient, data is gathered at step 216 indicating whether the patient has, or has not, in fact developed one or more bed sores. As another example, if algorithm 210 is being used to predict the development of a patient fall, data is gathered at step 216 indicating whether the patient has experienced a fall or not. Such “ground truth” data may be gathered in a variety of different manners, including, but not limited to, having a user enter such information directly into patient support apparatus 20 via one or more control panels 50, having patient support apparatus 20 query the EMR server 96 to see if the patient has developed a bed sore or fallen, or in other manners.

After gathering the data at step 216, controller 72 either processes the gathered data itself or it sends the data to an offboard computer device (e.g. remote computer 100) for processing. That is, as with steps 126 through 134 of algorithm 110, which may be either performed by controller 72 or an offboard computer device, so to may steps 218 through 224 of algorithm 210 be performed either by controller 72 or an offboard computer device (or multiple offboard computer devices, or by a combination of controller 72 and one or more offboard computer devices).

At step 218, either controller 72 or remote computer 100 adds the data gathered at step 216 to a database. The database contains similar sets of data that were gathered previously when the triggering action was still valid at step 214. That is, algorithm 210 repetitively loops through steps 214 through 222 until it discovers a correlation between the data in the database and the actual occurrence of the future event (the “ground truth”). The repetitive looping involves gathering data at step 216 both before the event happens and after the event is actually detected (“ground truth”). The database, which may be different from the database(s) used with algorithm 110, therefore contains readings of the data gathered at step 216 for each time the triggering action took place, as well as for each iteration through the cycle of algorithm 210 while the triggering event continued (e.g. was not cancelled), as well as one or more sets of data gathered after the event took place.

At step 220, either controller 72 or remote computer 100 analyzes the data in the database to determine what level of correlation exists, if any, between the data gathered at step 216 and the “ground truth,” as mentioned above. This correlation analysis may be performed in a variety of different manners, including, but not limited to, using one or more machine learning algorithms, such as, but not limited to, supervised learning methods, unsupervised learning methods, semi-supervised learning methods, reinforcement learning methods, feature learning methods, and/or self-learning methods. The model used by the machine learning algorithm may be based upon any one or more of the following: an artificial neural network, a decision tree, a support vector machine, a regression analysis, a Bayesian network, and/or a training model. Regardless of which specific machine learning algorithm and/or model is utilized, controller 72 or remote computer 100 carry out the analysis at step 220 in order to find a reliable correlation between at least one of the items of data gathered at step 216 and the “ground truth.” That is, controller 72 or remote computer 100 look for at least one item of the data (and likely a set of such data) that, over substantially all of the past times that the triggering action of step 214 took place, is a reliable indicator of the future event taking place.

For example, if algorithm 210 is configured to predict when a patient is about to exit from patient support apparatus 20, controller 72 or remote computer 100 is configured to look at all of the data gathered at step 216 and to see if any combination of one or more of the data items correlates to the occurrence of the patient actually exiting from patient support apparatus 20. Such analysis may determine, as an example, that a particular patient always exits after a certain amount of time on support deck 30 and/or after a certain amount of motion (as detected by force sensors 92q), and/or after one or more of the other sensors 92a-p detect one or more conditions, and/or a sequence of conditions.

After carrying out the analysis at step 220, controller 72 or remote computer 100 compares the correlation(s), if any, determined at step 220 to a threshold at step 222 (FIG. 11). In some embodiments, the threshold is configurable by the user, such as by navigating to a setting screen, or the like, on display 54 that allows the user to change the threshold. The threshold determines how reliable the correlations need to be before controller 72 will issue an alert of the possibility of the future event taking place (step 224). In some instances, the threshold may be 100 percent, in which case the alert will not be issued unless a perfect correlation can be found between one or more of the data items gathered at step 216 and the occurrence of the future event has been definitively established. In other instances, the threshold may be less so that an alert (or warning) of the future event taking place is issued when the probability of that event occurring is over the threshold.

If the correlation is determined at step 222 to be greater than the threshold (FIG. 11), controller 72 or remote computer 100 executes step 224. At step 224, controller 72 or remote computer 100 issues an alert to the user. The alert may take on different forms, including a local alert (local to patient support apparatus 20) that involves any combination of audio and/or visual indications on patient support apparatus 20. Still further, the alert may be a remote alert (to patient support apparatus 20) in which case the alert is either transmitted off of patient support apparatus 20 via network transceiver 84 (or another transceiver) and communicated to one or more servers (or it is issued by one or more servers, such as remote computer 100), and, in some embodiments, further communicated to one or more mobile electronic devices carried by one or more personnel of the healthcare facility. In some embodiments, patient support apparatus 20 and remote computer 100 are integrated into a caregiver assistance system and the alert is forwarded to one or more mobile electronic devices carried by one or more caregivers using the caregiver assistance system. One example of such a caregiver assistance system into which patient support apparatuses 20 and remote computer 100 may be integrated is disclosed in commonly assigned PCT patent application serial number PCT/US2020/039587 filed Jun. 25, 2020, and entitled CAREGIVER ASSISTANCE SYSTEM, the complete disclosure of which is incorporated herein by reference (including references incorporated by reference into the aforementioned PCT/US2020/039587 application). Still other types of alerts may be issued at step 224.

If remote computer 100 performs steps 218 through 224, it activates the alert at step 224 by sending a message to controller 72 (via transceiver 84) indicating that controller 72 should issue a local alert at step 224. If controller 72 performs steps 218 through 224, it activates the local alert and/or sends a message to remote computer 100 to activate the alert.

If controller 72 or remote computer 100 determines at step 222 that the correlation is less than the threshold, it returns to step 214 and continues to cycle through algorithm 210 in the manner previously described. In other words, when returning to step 214, controller 72 checks again to see that the triggering action that was analyzed in the previous iteration of step 214 is still in its triggering state (e.g. the patient is still present on support deck 30, exit detection system 88 is still armed, the patient still has a fall risk greater than a threshold, etc.). If it is, it continues to step 216 and proceeds in the manner previously described. If it is not, it returns to step 212 and waits until the triggering action takes place again.

It will be understood that when remote computer 100 is configured to carry out the analysis of step 220 (FIG. 11), it may be configured to utilize data gathered at step 216 from a plurality of patient support apparatuses 20 positioned within the same healthcare facility. Thus, when a triggering action occurs at step 214 for a particular patient support apparatus 20, the data utilized in step 220 of the algorithm 210 performed for that particular patient support apparatus 20 may comprise data that was gathered by remote computer 100 from other patient support apparatuses 20 when the same triggering action occurred for those other patient support apparatuses 20. In still other embodiments, remote computer 100 may carry out the analysis of step 220 using data from other healthcare facilities. In such embodiments, remote computer 100 may be positioned outside of the healthcare facility (and accessible to network 68 via the Internet), or remote computer 100 may be in communication with another remote computer via the Internet that has access to data gathered from patient support apparatuses 20 positioned at other healthcare facilities.

It will be understood that any patient support apparatus 20 may implement more than one instance of algorithm 210 for use in predicting the occurrence of different events. When done so, the data gathered at step 216 for the different events may be different, just as the data gathered at step 118 may be different for the different controls activated at step 114 of algorithm 110. Still further, patient support apparatuses 20 are user-customizable, in some embodiments, as to which algorithm 110 and/or 210 is to be implemented thereon, as well as to the number of instances of the algorithm 110 and/or 210 to executed thereon and as well as the content of the algorithms 110 and/or 210. In other words, the user is free to choose, for example, which setting(s) are to be subjected to algorithm 110 and/or which future events are to be subjected to algorithm 210.

It will also be understood that both algorithms 110 and 210 may be modified substantially from what is shown in FIGS. 4 and 11, respectively. As one example of such a modification, algorithm 210 may be modified such that controller 72 and/or remote computer 100 takes one or more actions automatically in response to a correlation being determined at step 222 that is greater than the threshold. These automatic actions may be in addition to, or in lieu of, the alert issued at step 224. Such automatic actions may include, for example, automatically implementing a mattress therapy via mattress 42 (or automatically recommending a mattress therapy to the caregiver) when the likelihood of the patient developing a bed sore has exceeded a threshold (for those embodiments where algorithm 210 is being used to predict the occurrence of a patient developing bed sores).

As with algorithm 110, the database analyzed at step 220 of algorithm 210 will likely include data from more sensors (and/or other sources) that than what controller 72 deems is necessary for predicting the future event. In other words, controller 72 will gather data at step 216 from a set of sensors (and/or other sources) that likely includes more data than is needed to accurately predict (or predict with a threshold-exceeding probability) the occurrence of the future event. This additional data is analyzed at step 220, but controller 72 may determine, in some embodiments, through repetitive iterations of algorithm 210 that this data does not have sufficient predictive power to predict the future event. Accordingly, in such embodiments, controller 72, after populating the database sufficiently to determine the predictive power of the data from step 216 for the occurrence of the future event, no longer uses the data from step 216 that has insufficient predictive power for the occurrence of the future event. Stated still more simply, controller 72 gathers data from a relatively large set of sensors (and/or other sources) at step 216, but only uses the outputs of a subset of those sensors (and/or other sources) at step 222 when determining whether to issue the alert at step 224. As noted, this is because controller 72 searches for correlations between the entire set of data gathered at step 216 and the future event, but such correlations typically only exist for a subset of this data.

In some embodiments, the analysis carried out at steps 218 and 220 is the development (or improvement) of an algorithm for predicting the occurrence of the future event before the future event actually happens. In such embodiments, if the analysis is carried out by remote computer 100, remote computer 100 transmits to patient support apparatus 20 a new algorithm after the database has been sufficiently populated with enough data for remote computer 100 to conclude that the new algorithm is sufficiently reliable for predicting the occurrence of the future event. In such embodiments, controller 72 activates the new algorithm whenever the triggering action of step 214 occurs and uses the new algorithm to determine whether or not the probability of the future event occurring has reached such a level (e.g. more than the threshold at step 222) that an alert should be issued (e.g. step 224).

As mentioned above, the new algorithm may not utilize all of the outputs from the sensors that is collected at step 216. That is, either controller 72 (or remote computer 100) uses data from a relatively large set of sensors (and/other sources) to determine if an algorithm can be formulated with sufficient predict power (done at steps 126-130). To the extent such an algorithm can be formulated, it typically will utilize only a subset of the data analyzed at steps 126-130. Accordingly, controller 72 (or remote computer 100) uses a broad set of information in its search for a sufficiently reliable algorithm and when that algorithm is found, it typically will only use a subset of that broad set of information when implementing the new algorithm for predicting and/or automatically selecting the users preferred setting(s).

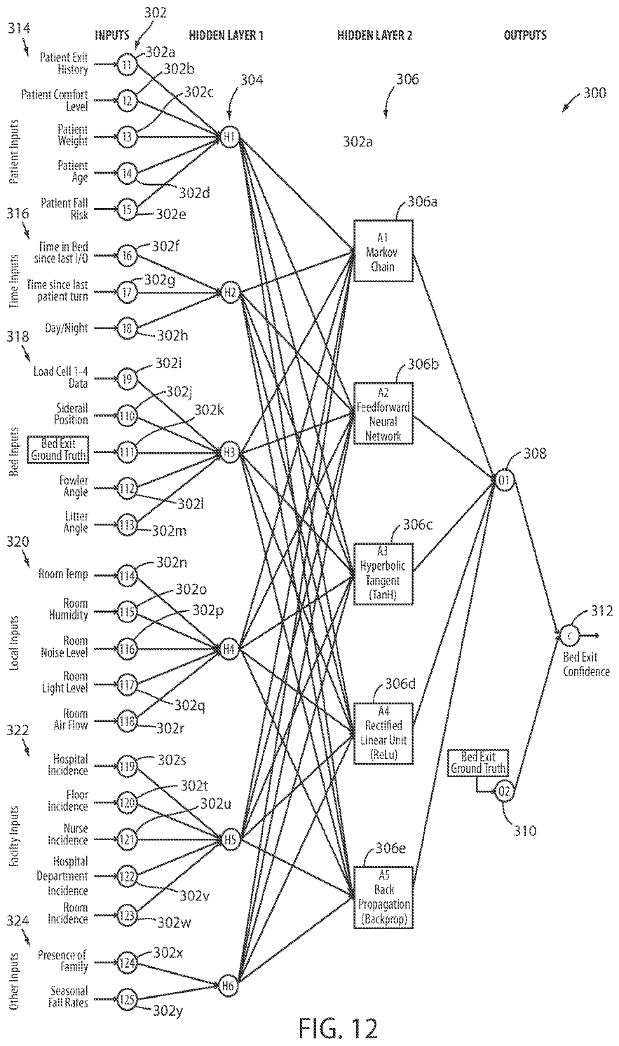

FIG. 12 illustrates one example of a neural network 300 that may be utilized by controller 72 and/or remote computer 100 when performing any of steps 126-130 of algorithm 110 and/or any of steps 218-222 of algorithm 210. In the particular example shown in FIG. 12, neural network 300 is specifically tailored for predicting when a patient may be about to exit from patient support apparatus 20. It will be understood, however, that neural network 300 may be modified for predicting other events, as well as for predicting any one or more of the user-preferred settings discussed above with respect to algorithm 110.

Neural network 300 includes a plurality of inputs 302, a plurality of first hidden layer nodes 304, a plurality of second hidden layer nodes 306, a first output 308, a second output 310, and a confidence score 312. The inputs 302 include a set of patient inputs 314 comprising inputs 302a-e, a set of time inputs 316 comprising inputs 302f-h, a set of patient support apparatus inputs 318 comprising inputs 302i-m, a set of room inputs 320 comprising inputs 302n-r, a set of facility inputs 322 comprising inputs 302s-w, and a set of other inputs 324 comprising inputs 302x-y. It will be understood that both the sets of inputs 314-324 and the individual inputs 302 within those sets may be changed from what is shown in FIG. 12.

Each of these inputs 302 will now be described with more detail. Input 302a comprises a history of the exits (from patient support apparatus 20) of the patient currently assigned to patient support apparatus 20. This history may be stored onboard patient support apparatus 20 (e.g. in memory 74) and/or it may be stored on remote computer 100. Such a history may be generated from recording the outputs from exit detection system 88, which indicate when the patient has left patient support apparatus 20. Controller 72 may determine that a particular history of patient exits corresponds to a particular patient in multiple manners. In one such manner, controller 72 communicates with EMR server 96 and/or another server on network 68 to determine which patient is assigned to patient support apparatus 20 (and when that assignment changes) and controller 72 then aggregates the detected exits to that particular patient. In another manner, new patient information is entered into patient support apparatus 20 by a caregiver whenever a new patient is assigned to patient support apparatus 20 and controller 72 simply uses that entered information to aggregate detected exits to a particular patient. Still other manners may be used.

Input 302b refers to a patient comfort and/or mobility level. In some embodiments, this level is determined by how active the patient is while positioned on support deck 30 (as detected by force sensors 92q). Methods of determining this activity level include monitoring the amount of movement of the patient over time. In some embodiments, any one or more of the methods disclosed in any of the following commonly assigned patent applications may be used to determine a patient comfort and/or mobility level: U.S. Pat. No. 9,814,410 issued Nov. 14, 2017 and entitled PERSON SUPPORT APPARATUS WITH POSITION MONITORING; and U.S. patent application Ser. No. 15/809,351 filed Nov. 10, 2017, by inventors Patrick Lafleche et al. and entitled PATIENT SUPPORT APPARATUSES WITH MOBILITY ASSESSMENT, the complete disclosures of which are both incorporated herein by reference.

Input 302c refers to the patient's weight. This input is determined by summing the outputs from force sensors 92q and subtracting the tare weight while the patient is positioned on support deck 30. Alternatively, the patient's weight may be read from EMR server 96. In some embodiments, force sensors 92q may be part of a scale system of the type disclosed in commonly assigned U.S. patent application Ser. No. 16/992,515 filed Aug. 13, 2020, by inventors Kurosh Nahavandi et al. and entitled PATIENT SUPPORT APPARATUSE WITH EQUIPMENT WEIGHT LOG, the complete disclosure of which is incorporated herein by reference. Such a scale system determines the patient's weight and may be incorporated into patient support apparatus 20, thus supplying controller 72 with the patient's weight for input 302c.

Input 302d refers to the patient's age. This information may be determined by controller 72 sending a query to EMR server 96, or another server, and/or it may be determined by having a caregiver enter this information directly into patient support apparatus 20 via a control panel 50.

Input 302e refers to the patient's fall risk. In most embodiments, input 302e refers to a score generated as part of a fall risk assessment, such as, for example, a score of the Braden scale. This information may be determined by controller 72 sending a query to EMR server 96, or another server, and/or it may be determined by having a caregiver enter this information directly into patient support apparatus 20 via a control panel 50.

Input 302f refers to the amount of time since the patient last evacuated his or her bowels and/or his or her bladder. This information may be determined by controller 72 sending a query to EMR server 96, or another server, and/or it may be determined by having a caregiver enter this information directly into patient support apparatus 20 via a control panel 50.

Input 302g refers to the amount of time since the patient was last turned. This information may be obtained from controller 72 which, in some cases, controls mattress 42 to assist the caregiver in turning the patient. In those situations where the patient is turned without using mattress 42, controller 72 obtains this information by sending a query to EMR server 96, or another server, and/or it may be determined by having a caregiver enter this information directly into patient support apparatus 20 via a control panel 50.

Input 302h refers to the time of day and/or the calendar day. In some embodiments, patient support apparatus 20 includes an onboard clock and calendar that keeps track of the current time of day and current calendar day. In some such embodiments, patient support apparatus 20 may include an onboard clock that operates in any of the manners disclosed in commonly assigned U.S. patent application Ser. No. 15/642,621 filed Jul. 6, 2017, by inventors Anuj K. Sidhu et al. and entitled PATIENT SUPPORT APPARATUSES WITH CLOCKS, the complete disclosure of which is incorporated herein by reference. Still other manners for obtaining the time of day and/or calendar day may be implemented.

Input 302i refers to the outputs from force sensors 92q. Input 302j refers to the outputs from siderail sensors 92a. Input 302k refers to the ground truth, which in the example shown in FIG. 12, refers to an indication of whether or not the patient has actually exited from patient support apparatus 20. In some embodiments, this information comes from exit detection system 88. In other embodiments, this information may come from other sources such as, but not limited to, one or more video monitoring systems and/or one or more off-board exit detection systems.

Input 302l refers to the angle of head section 44 and comes from one of deck angle sensors 92b. Input 302m refers to the angle (with respect to horizontal and/or the floor) of litter frame 28 and comes from litter tilt angle sensor 92e.

Input 302n refers to a current temperature of the room in which patient support apparatus 20 is currently positioned and comes from room temperature sensor 92j. Input 302o refers to a current humidity of the room in which patient support apparatus 20 is currently positioned and comes from room humidity sensor 92k. Input 302p refers to an amount of ambient noise in the room of patient support apparatus 20 and comes from room sound sensor 92l. Input 302q refers to an amount of light in the room of patient support apparatus 20 and comes from light sensor 92n. Input 302r refers to an amount of air flow in the room of patient support apparatus 20 and comes from room air flow sensor 92m.

Input 302s refers to the number of times and/or frequency at which the event that algorithm 110 or 210 is being used to anticipate or predict occurs within the healthcare facility. In the particular example shown in FIG. 12, input 302s refers to the number of times and/or frequency at which a patient exits from a patient support apparatus 20 for the healthcare facility in which patient support apparatus 20 is positioned. This data, in at least one embodiment, is gathered by remote computer 100. In such embodiments, every time a patient support apparatus 20 detects an exit of a patient (via its onboard exit detection system 88), it reports that to remote computer 100. Remote computer 100 saves and compiles this data from all of the patient support apparatuses 20 and makes it available to those controllers 72 who are executing the specific neural network 300 shown in FIG. 12.

Input 302t refers to the number of times and/or frequency at which the event that algorithm 110 or 210 is being used to anticipate or predict occurs on the particular floor in which patient support apparatus 20 is currently positioned. In the particular example shown in FIG. 12, input 302s refers to the number of times and/or frequency at which a patient exits from a patient support apparatus 20 that is positioned on the same floor of the healthcare facility as patient support apparatus 20. As with the data for input 302s, this data, in at least one embodiment, is gathered by remote computer 100. In such embodiments, every time a patient support apparatus 20 detects an exit of a patient (via its onboard exit detection system 88), it reports not only the exit detection to remote computer 100, but also its location within the healthcare facility (or it reports a location ID that can be used by remote computer 100 to determine the location of patient support apparatus 20). This location information may be gathered by patient support apparatuses 20 in a variety of different manners, including, but not limited to, any of the location-determining methods disclosed in commonly assigned PCT patent application serial number PCT/US2020/039587 filed Jun. 25, 2020, by inventors Thomas Durlach et al. and entitled CAREGIVER ASSISTANCE SYSTEM, the complete disclosure of which has already been incorporated herein by reference. Remote computer 100 is therefore able to determine which patient support apparatuses 20 are positioned on which floors of the healthcare facility and to arrange the data is receives and saves according to the different floors of the healthcare facility.

Input 302u refers to the number of times and/or frequency at which the event that algorithm 110 or 210 is being used to anticipate or predict occurs for the same caregiver who is assigned to patient support apparatus 20. In the particular example shown in FIG. 12, input 302u refers to the number of times and/or frequency at which a patient exits from a patient support apparatus 20 whose patient is assigned to the same caregiver. As with the data for inputs 302s and 302t, this data, in at least one embodiment, is gathered by remote computer 100. In such embodiments, remote computer 100 is configured to determine the caregiver assigned to each patient support apparatus 20 so that every time a patient support apparatus 20 reports an patient exit, remote computer 100 can associate that patient exit with a particular caregiver. As was mentioned previously, this caregiver assignment information may be gathered by remote computer 100 in a variety of different manners, including, but not limited to, any of the caregiver-determining methods used by the caregiver assistance application disclosed in commonly assigned PCT patent application serial number PCT/US2020/039587 filed Jun. 25, 2020, by inventors Thomas Durlach et al. and entitled CAREGIVER ASSISTANCE SYSTEM, the complete disclosure of which has already been incorporated herein by reference.

Input 302v refers to the number of times and/or frequency at which the event that algorithm 110 or 210 is being used to anticipate or predict occurs within the same department or ward that patient support apparatus 20 is part of. In the particular example shown in FIG. 12, input 302v refers to the number of times and/or frequency at which a patient exits from a patient support apparatus 20 that is positioned within the same ward or department of the healthcare facility. As with the data for inputs 302s-u, this data, in at least one embodiment, is gathered by remote computer 100. In such embodiments, remote computer 100 is configured to determine the department or ward assigned to each patient support apparatus 20 so that every time a patient support apparatus 20 reports an patient exit, remote computer 100 can associate that patient exit with a particular ward or department of the healthcare facility. This information may be input directly into remote computer 100 by an authorized representative of the healthcare facility, or it may be obtained by remote computer 100 querying one or more servers on network 68 that have this information available.

Input 302w refers to the number of times and/or frequency at which the event that algorithm 110 or 210 is being used to anticipate or predict occurs within the same room that patient support apparatus 20 is part of. In the particular example shown in FIG. 12, input 302v refers to the number of times and/or frequency at which a patient exits from a patient support apparatus 20 that is positioned within the same room of the healthcare facility. This data may be gathered by remote computer 100 in any of the same manners discussed above for inputs 302s-v.

Input 302x refers to the presence of the patient's friend or family member in the same room as the patient support apparatus 20 to which the patient is assigned. In some embodiments, the friends or family members are detected by badges, tags, smart phone apps, or other devices that are adapted to automatically communicate and/or respond to interrogations from patient support apparatus 20 when they are positioned within the same room as patient support apparatus 20. Alternatively, or additionally, the presence of a friend or family member may be detected by an app on the friend or family members cell phone that requests input from the friend or family member regarding their visit to the healthcare facility. In such embodiments, the app communicates with one or more servers on network 68 and remote computer 100 retrieves this information from those one or more servers. Still other manners of detecting the patient's friend or family members in the room and/or healthcare facility may be used.

Input 302y refers to the seasonal rates at which the event that algorithm 110 or 210 is being used to anticipate or predict occurs for that patient support apparatus 20. In the particular example shown in FIG. 12, input 302y refers to the rate or frequency at which a patient exits from patient support apparatus 20 during the current season, or during another slice of time. As with the data for inputs 302s, this data, in at least one embodiment, is gathered by remote computer 100. This data may be gathered by remote computer 100 in any of the same manners discussed above for inputs 302s-v.

After receiving all of the inputs 302, either controller 72 (or remote computer 100) executes the computations of neural network 300 (FIG. 12). These computations include the first hidden layer 304 which, as shown, groups together the inputs according to their respective sets 314, 316, 318, 320, 322, and 324. The outputs of the first hidden layer are forwarded to the second hidden layer, which includes nodes 306a-e. Node 306a is, in the illustrated embodiments, a Markov chain node. Node 306b is a feedforward neural network node; node 306c is a hyperbolic tangent node; node 306d is a rectified linear unit node; and node 306e is a back propagation node. It will be understood that any of the nodes 306a-e may be changed from what is illustrated in FIG. 12.

For both hidden layers 304 and 306, the number of nodes, the content of the nodes, the inputs to those nodes, and the outputs of the nodes may be modified from what is shown in FIG. 12, including, but not limited to, the substitutions, additions, and/or deletions of one or more of these nodes with other nodes and/or other types of nodes not shown in FIG. 12.

The outputs from the hidden layers 304, 306, etc. are processed by controller 72 and/or remote computer 100 to product an output 308 that is compared to the ground truth 310 in order to determine a confidence level of neural network 300. In the illustrated embodiment, the confidence level refers to an acceptable accuracy at which neural network 300 is able to predict the exiting of a patient from patient support apparatus 20 before the patient actually exits. This confidence level 312 is used at steps 134 and/or 222. That is, at these steps controller 72 or remote computer 100 determines if the confidence level has exceeded the threshold defined in these steps. If so, neural network 300 may be used in the future to predict a future event, such as the selection of a particular setting (e.g. algorithm 110) or the prediction of some other type of future event (algorithm 210).

As was noted, although FIG. 12 illustrates a specific neural network 300 used for carrying out a specific event prediction (patient exiting from patient support apparatus 20), it will be understood that controller 72 and/or remote computer 100 may use not only other types of neural networks at steps 128 and 220, but also other types of machine learning algorithms.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A patient support apparatus comprising:
a frame;
a support surface adapted to support a patient thereon, the support surface supported on the frame;
a control adapted to be activated by a user of the patient support apparatus;
a controller in communication with the control, the controller adapted to perform a function of the patient support apparatus when the control is activated by the user, the function being performed in a plurality of different manners based upon a setting selectable by the user, wherein the controller is further adapted to record over time setting data indicating the setting selected by the user when the function is performed;
a transceiver adapted to transmit the setting data to a computing device located off-board the patient support apparatus, the computing device adapted to analyze the setting data and to determine a user-preferred setting when the function is performed; and
wherein the controller is further adapted to receive a message back from the computing device indicating the user-preferred setting and to automatically select the user-preferred setting when the user activates the control.

2. The patient support apparatus of claim 1 wherein the function is an exit alert issued when the patient moves toward exiting from the support surface, and the setting is a sensitivity level for the exit alert.

3. The patient support apparatus of claim 1 wherein the support surface includes a backrest section adapted to pivot over a range of orientations, the function is a lockout function adapted to prevent pivoting of the backrest section below a specific angle, and the setting is the specific angle.

4. The patient support apparatus of claim 1 further comprising an inflatable bladder in communication with the controller, and wherein the function is a therapy function administered by the inflatable bladder and the setting is at least one of a duration, a frequency, or a rotational angle related to the therapy function.

5. The patient support apparatus of claim 1 further comprising a sensor, wherein the controller is further adapted to take multiple sets of readings from the sensor and record the sets of readings, each set of the multiple sets of readings including readings taken both before and after an occurrence of an event associated with the patient support apparatus; and wherein the controller is further adapted to transmit the sets of readings to the computing device.

6. The patient support apparatus of claim 5 wherein the controller is further adapted to receive an algorithm back from the computing device for predicting a future occurrence of the event.

7-10. (canceled)

11. A patient support apparatus system comprising:
a patient support apparatus comprising:
(a) a frame;
(b) a support surface adapted to a support a patient thereon;
(c) a sensor;
(d) a controller adapted to take multiple sets of readings from the sensor and record the sets of readings, each set of the multiple sets of readings including readings taken both before and after an occurrence of an event associated with the patient support apparatus; and
(e) a transceiver; and
a computing device positioned remotely from the patient support apparatus and in communication with the transceiver, the computing device adapted to receive the sets of readings from the patient support apparatus and to analyze the sets of readings to determine an algorithm for predicting a future occurrence of the event using future readings from the sensor.

12. The patient support apparatus system of claim 11 wherein at least one of the following is true:
(a) the event is the patient exiting from the support surface and the sensor comprises a plurality of force sensors adapted to detect downward forces exerted on the support surface;
(b) the event is the patient contracting ventilator associated pneumonia, the support surface includes a backrest section adapted to pivot over a range of orientations, and the sensor is adapted to measure an angular orientation of the backrest section; or
(c) the event is the patient contracting a bed sore, patient support apparatus further comprises an inflatable bladder in communication with the controller and adapted to administer a therapy function to the patient, and the sensor is adapted to measure at least one of a duration, a frequency, or a rotational angle related to the therapy function.

13-17. (canceled)

18. The patient support apparatus system of claim 11 wherein the controller is further adapted to execute a function using the sensor and a second algorithm.

19. The patient support apparatus system of claim 18 further comprising a plurality of additional sensors, wherein the controller is adapted to perform the following: receive an additional set of readings from the additional sensors, record the additional set of readings, transmit the additional set of readings to the computing device, receive an improved second algorithm back from the computing device, and use the improved second algorithm when performing the function, wherein the improved second algorithm uses readings from at least one of the plurality of additional sensors.

20. (canceled)

21. The patient support apparatus system of claim 19 wherein the plurality of additional sensors includes at least two of the following: a siderail sensor adapted to detect a position of a siderail of the patient support apparatus, an angle sensor adapted to detect an angle of a pivotable backrest section of the patient support apparatus, an angle sensor adapted to detect an angle of a litter frame on which the support surface is supported, a light sensor adapted to detect an amount of ambient light in a room in which the patient support apparatus is positioned, a sound sensor adapted to detect an amount of sound in the room in which the patient support apparatus is positioned, or a clock adapted to detect a current time.

22. The patient support apparatus system of claim 19 wherein the computing device is adapted to use a neural network to generate the improved second algorithm and the computing device is further adapted to use at least two of the following as inputs into the neural network: a patient fall history, a patient weight, a patient age, a patient fall risk assessment, a time of day, an amount of time since the patient last exited from the patient support apparatus, and a calendar date.

23. (canceled)

24. The patient support apparatus system of claim 11 wherein the controller is further adapted to perform a function of the patient support apparatus when a control is activated by a user, the function being performed in a plurality of different manners based upon a setting selectable by the user, wherein the event is the selection of the setting by the user, and wherein the algorithm is adapted to predict a future setting in response to the user activating the control.

25. A patient support apparatus system comprising:
a plurality of patient support apparatuses, each one of the plurality of patient support apparatuses comprising:
(a) a frame;
(b) a support surface adapted to a support a patient thereon;
(c) a plurality of sensors;
(d) a controller adapted to take a set of readings from the plurality of sensors and use a first subset of the set of readings in an algorithm for performing a function of the patient support apparatus, the first subset excluding readings from at least one sensor in the plurality of sensors, wherein the controller is further adapted to record the set of readings; and
(e) a transceiver; and
a computing device positioned remotely from the patient support apparatus and in communication with the transceiver, the computing device adapted to receive the set of readings from the plurality of patient support apparatuses and to analyze the set of readings to determine an improved algorithm for use by each of the plurality of patient support apparatuses when performing the function.

26. The patient support apparatus system of claim 25 wherein the controller is further adapted to receive the improved algorithm back from the computing device and to use the improved algorithm when performing the function, wherein the improved algorithm uses a second subset of the readings from the plurality of sensors, the second subset being different from the first subset.

27. The patient support apparatus system of claim 26 wherein the second subset of the readings does not exclude readings from the at least one sensor in the plurality of sensors.

28. The patient support apparatus system of claim 26 wherein the first subset includes at least one reading from a sensor not included in the second subset.

29. The patient support apparatus system of claim 26 wherein the support surface includes a backrest section adapted to pivot over a range of orientations, the function is a lockout function adapted to prevent pivoting of the backrest section below a specific angle, and the first subset includes readings from an angle sensor adapted to detect an angular orientation of the backrest section.

30. The patient support apparatus system of claim 26 wherein each of the plurality of patient support apparatuses further comprises an inflatable bladder in communication with the controller, and wherein the function is a therapy function administered by the inflatable bladder, the first subset includes readings from an angle sensor adapted to detect an rotational angle related to the therapy function, and the set of readings includes readings taken from at least one of a scale system adapted to detect a weight of the patient or a timer adapted to measure an amount of time since the therapy function was previously performed.

31. (canceled)

32. The patient support apparatus system of claim 26 wherein the function is an exit alert issued when the patient moves toward exiting from the support surface, the first subset includes reading from a plurality of force sensors adapted to detect downward forces exerted on the support surface, and the plurality of sensors includes at least two of the following: a siderail sensor adapted to detect a position of a siderail of a respective patient support apparatus, an angle sensor adapted to detect an angle of a pivotable backrest section of the respective patient support apparatus, an angle sensor adapted to detect an angle of a litter frame on which the support surface is supported, a light sensor adapted to detect an amount of ambient light in a room in which the respective patient support apparatus is positioned, a sound sensor adapted to detect an amount of sound in the room in which the respective patient support apparatus is positioned, or a clock adapted to detect a current time.

33-36. (canceled)
